SEQUENCE LISTING (41 sequences)

SEQ ID NO: 1 - protein, 1049 aa, Bifidobacterium adolescentis
MADTAELAIVHATTASASWLTDPTVFAANRKPAHSSHRYVIGETSEPKQSLDGEWKVRIEQARNVDVESAPFAAVDFEDGDFGAIEVPGHLQMAGYLKNKYVNIQYPWDGHEDPQAPNIPENNHVAIYRRRFALDAQLARTLENDGTVSLTFHGAATAIYVWLDGTFVGYGEDGFTPSEFDVTEALRNGNGNAADSPEAEHTLTVACYEYSSASWLEDQDFWRLHGLFRTVELAAQPHTHVETVQLEADYTAADTAGTADTAELNAALTLRNSADAMTIESTLRDGDGNVVWESTQACNGEIALNSGKMTNIAPWSAESPTLYTLTVRVVGHDGAIIETVTQKIGFRTFRIENGIMTLNGKRIVFKGADRHEFDAKRGRAITREDMLSDVVFCKRHNINAIRTSHYPNQEYWYDLCDEYGLYLIDETNMETHGTWVANNVERPEDGIPGSRPEWEGACVDRINSMMRRDYNHPSVLIWSLGNESSAGEVFRAMYRHAHTIDPNRPVHYEGSVHMREFEDVTDIESRMYAHADEIERYLNDGSPAHTDGPKKPYISCEYMHAMGNSCGNMDEYTALERYPMYQGGFIWDFIDQAIETKLPDGTTRMCYGGDFGDRPSDYEFSGDGLLFADRTPSPKAQEVKQLYANVKIVVSVDEARITNDNLFVSTGDYRFVLRILADGKPVWSTTRRFDVAAGESASFEVDWPVDDYRSNAEELVLEVSQQLGNACDWAPAGYELAFGQCVVAGAKTTADAVDAAGAPADGTVTLGRWNAGVRGQGREALFSRTQGGMVSYTFGEREFVLRRPSITTFRPLTDNDRGAGHAFERAAWAVAGKYARCVDCAIANRGENAVEATYTYELAIPQRTKVTVRYVADTAGLVSLDVEYPGEKNGDLPTIPAFGIEWALPVEYANLRFYGAGPEETYADRRHAKLGVWSTTAGDDCAPYLLPQETGNHEDVRWAEITDDSGHGVRVKRGAGAKPFAMSLLPYSSTMLEEALHQDELPKPRHMFLRLLAAQMGVGGDDSWMSPVHEQYQLPADQPLSLNVQLKLF

SEQ ID NO: 2 - protein, 625 aa, Lactobacillus sakei
MQPNIQWLDTPAVFRVGQLPAHSDHRYYATLAEMAQQQSSFEQSLNGTWQFHYSVNAASRPKSFYELAFDAQDFEPITVPQHIELAGYEQLHYINTMYPWEGHYYRRPAFSTSDDKQHLGMFSEADYNPVGSYLHHFDLTPALRNQRVIIRFEGVEQAMYVWLNGQFIGYAEDSFTPSEFDLTPYLKETDNCLAVEVHKRSSAAFIEDQDFFRFFGIFRDVKLLAKPRTHLEDLWVIPEYDVVQQTGQVKLRLQFSGDENRVHLRIRDQHQIILTADLTSAAQVNGLYKMPELVQAWSNQTPNLYTLELEVVDQAGETIEISQQPFGFRKIEIKDKVMLLNGKRLVINGVNRHEWHPETGRTITAEDEAWDIACMQRNHINAVRTSHYPDRLSFYNGCDQAGIYMMAETNLESHGSWQKMGAVEPSWNVPGSYDEWEAATLDRARTNFETFKNHVSILFWSLGNESYAGSVLEKMNAYYKQQDPTRLVHYEGVFRAPEYKATISDVESRMYATPAEIKAYLDNAPQKPFILCEYMHDMGNSLGGMQSYIDLLSQYDMYQGGFIWDFIDQALLVTDPVTGQRELRYGGDFDDRPSDYEFSGDGLVFATRDEKPAMQEVRYYYGEHK

SEQ ID NO: 3 - protein, 330 aa, Lactobacillus sakei
MKNQQCRRLDTIMANTNKRLAVIFGDVTLGLKGPDFHYLFSYQTGGPESLRIQGKEWLYRSPKPTFWRATTDNDRGNQFPLKSGMWLAADQFIACQSITVAIDGQTIPLPIAPENNRYSGQETAQEVTVTYTYQTITTPQTTVEVSYTIQASGKIRVAVTYHGQAGLPSLPVFGLRFVMPTPATRFIYQGLSGETYPDRMAGGMAGEYEVTGLPVTPYLVPQDCGVHMATDWVTIYRQAVLDNRLREPVETGLKFKMVDQPFAFSCLPYTAEELENATHHSELPAPHRTVLSLLGAVRGVGGIDSWGSDVEAAYQIDATQDHHLEFEISF

SEQ ID NO: 4 - protein, 1049 aa, Bifidobacterium adolescentis
MADTAELAIVHATTASASWLTDPTVFAANRKPAHSSHRYVIGETSEPKQSLDGEWKVRIEQARNVDVESAPFAAVDFEDGDFGAIEVPGHLQMAGYLKNKYVNIQYPWDGHEDPQAPNIPENNHVAIYRRRFALDAQLARTLENDGTVSLTFHGAATAIYVWLDGTFVGYGEDGFTPSEFDVTEALRNGNGNAADSPEAEHTLTVACYEYSSASWLEDQDFWRLHGLFRTVELAAQPHTHVETVQLEADYTAADTAGTADTAELNAALTLRNPADAMTIESTLRDGDGNVVWESTQACNGEIALNSGKMTNIAPWSAESPTLYTLTVRVVGHDGAIIETVTQKIGFRTFRIENGIMTLNGKRIVFKGADRHEFDAKRGRAITREDMLSDVVFCKRHNINAIRTSHYPNQEYWYDLCDEYGLYLIDETNMETHGTWVANNVERPEDGIPGSRPEWEGACVDRINSMMRRDYNHPSVLIWSLGNESSAGEVFRAMYRHAHTIDPNRPVHYEGSVHMREFEDVTDIESRMYAHADEIERYLNDGSPAHTDGPKKPYISCEYMHAMGNSCGNMDEYTALERYPMYQGGFIWDFIDQAIETKLPDGTTRMCYGGDFGDRPSDYEFSGDGLLFADRTPSPKAQEVKQLYANVKIAVSVDEARITNDNLFVSTGDYRFVLRILADGKPVWSTTRRFDVAAGESASFEVDWPVDDYRSNAEELVLEVSQQLGNACDWAPAGYELAFGQCVVAGAKTTADAVDAAGAPADGTVTLGRWNAGVRGQGREALFSRTQGGMVSYTFGEREFVLRRPSITTFRPLTDNDRGAGHAFERAAWAVAGKYARCVDCAIANRGENAVEATYTYELAIPQRTKVTVRYVADTAGLVSLDVEYPGEKNGDLPTIPAFGIEWALPVEYANLRFYGAGPEETYADRRHAKLGVWSTTAGDDCAPYLLPQETGNHEDVRWAEITDDSGHGVRVKRGAGAKPFAMSLLPYSSTMLEEALHQDELPKPRHMFLRLLAAQMGVGGDDSWMSPVHEQYQLPADQPLSLNVQLKLF

SEQ ID NO: 5 - protein, 626 aa, Lactobacillus amylovorus
MKANIKWLDDPEVFRINQLPAHSDHPFYKDYREWQNHSSSFKQSLNGAWQFHFSKDPQSRPIDFYKRSFDSSSFDTIPVPSEIELNGYAQNQYTNILYPWESKIYRKPAYTLGRGIKDGDFSQGKDNTVGSYLKHFDLNPALAGHDIHIQFEGVERAMYVYLNGHFIGYAEDSFTPSEFDLTPYIQAKDNILAVEVFKHSTASWLEDQDMFRFSGIFRSVELLALPRTHLMDLDIKPTVVNDYHDGVFNAKLHFMGKTSGNVHVLIEDIDGKTLLNKKLPLKSTVEIENETFANVHLWDNHDPYLYQLIIEVHDQDGKLVELIPYQFGFRKIEITKDHVVLLNGKRLIINGVNRHEWDAKRGRSITLADMKQDIATFKHNNINAVRTCHYPNQIPWYYLCDQNGIYMMAENNLESHGTWQKLGQVEATSNVPGSIPEWREVVVDRARSNYETFKNHTAILFWSLGNESYAGSNIAAMNKLYKDHDSSRLTHYEGVFHAPEFKKEISDLESCMYLPPKEAEEYLQNPKKPLVECEYMHDMGTPDGGMGSYIKLIDKYPQYMGGFIWDFIDQALLVHDPVSGQDVLRYGGDFDDRHSDYEFSGDGLMFADRTPKPAMQEVRYYYGLHK

SEQ ID NO: 6 - protein, 316 aa, Lactobacillus amylovorus
MAYTNNLHVVYGEASLGVNGQDFAYLFSYERGGLESLKIKDKEWLYRTPTPTFWRATTDNDRGSGFNQKAAQWLGADMFTKCVGIHVQVDDHRFDELPVAPINNQFSNQEFAHEVKVAFDYETLTTPATKVKIIYNINDFGHMTITMHYFGKKGLPPLPVIGMRFIMPTKAKSFDYTGLSGETYPDRMAGAERGTFHIDGLPVTKYLVPQENGMHMQTNELVITRNSTQNNADKDGDFSLKITQTKQPFNFSLLPYTAEELENATHIEELPLARRSVLVIAGAVRGVGGIDSWGSDVEEQYHIDPEQDHEFSFTLN

SEQ ID NO: 7 - protein, 1052 aa, Bifidobacterium bifidum
MNTTDDQRKNGDPIVSPSIPTTAWLADPRVYAVHRLDAHSDHACWSRSPVDGESTDLRQSLDGEWRVRVETAPTGRFPDGTSDGPDWISDVSPLFAAPGFDDSSFSRVQVPSHLETAGLLAPQYVNVQYPWDGHEDPKAPAIPEHGHVAVYRREFDADGEVAQAVREGRPVTLTFQGAATAIYVWLNGSFIGYAEDSFTPSEFDVTDAIKVDGNVLAVACYEYSSASWLEDQDFWRLHGLFRSVELNARPAAHVADLHADADWDLATSRGSLSLDVLIDGAANAATADFALRDKNGTIVWRTATKADGTLHAEAEIDDAAPWSAERPDLYELSVTLLDADGKVLETARTRIGFRHVAIEDGILKLNGKRLVFRGVNRHEFDCRRGRAITEEDMLWDIRFMKRHNINAVRTSHYPNQSRWYELCDEYGIYLIDETNLETHGSWNSPGDIPVGTSVPGDDEAWLGACIDRLDSMILRDRNHPSVLVWSLGNESYAGEVLKAMSAHAHQLDPGRPVHYEGVNWNHAYDGISDFESRMYAKPAEIQDWLEHGDERGEASKPFVSCEYMHAMGNSCGGLSEFIDLERYERYSGGFIWDYIDQGLVQRLPDGSERLSVGGEWGDRPTDYEFVGNGIVFADRTPSPKAQEVKQLYSPVKLAPDGHGVTIENRNLFAGTDGYVFAARLLEDGHEIWHADYRFDVAAGDTQHHDIAFPDIDADGDTREVTYEVDLLLAEATAWAPAGYELAFGQLTGTLNPEQDITETSHDDDGRATRTLSRWNAGIRRDDEEILLSRTQGGIVSWKRDDREMVIRRPELVTFRPLTDNDRGNHSGFDRAAWFAAGRYAIVTETKIHESDDGLVAEYQYELADPNHTPVSVTYHVNSDMRMQLTVEYPGNATDMASLPAFGIEWELPGEYDRLRYYGPGPEETYRDRKQGGKLGIWDATAKASMAPYLMVQETGSHEDVRWLEATDIQGHGLRVTQRGDRHFTASLLPWNTYTIEAARRHEDLPKPRHNYLRLLAAQMGVGGDDSWGAPVHTAYQLPAGRPLTLDVNLELI

SEQ ID NO: 8 - protein, 1052 aa, Bifidobacterium bifidum
MNTTDDQRKNGDPIVSPSIPTTAWLADPRVYAVHRLDAHSDHACWSRSPVDGESTDLRQSLDGEWRVRVETAPTGRFPDGTSDGPDWISDVSPLFAAPGFDDSSFSRVQVPSHLETAGLLAPQYVNVQYPWDGHEDPKAPAIPEHGHVAVYRREFDADGEVAQAVREGRPVTLTFQGAATAIYVWLNGSFIGYAEDSFTPSEFDVTDAIKVDGNVLAVACYEYSSASWLEDQDFWRLHGLFRSVELNARPAAHVADLHADADWDLATSRGSLSLDVLIDGAANAATADFALWDKNGTIVWHIVTKADGTLHAEAEIDDAAPWSAERPDLYELSVTLLDADGKVLETARTRIGFRHVAIEDGILKLNGKRLVFRGVNRHEFDCRRGRAITEEDMLWDIRFMKRHNINAVRTSHYPNQSRWYELCDEYGIYLIDETNLETHGSWNSPGDIPVGTSVPGDDEAWLGACIDRLDSMILRDRNHPSVLVWSLGNESYAGEVLKAMSAHAHRLDPGRPVHYEGVNWNHAYDGISDFESRMYAKPAEIQDWLEHGDERGEASKPFVSCEYMHAMGNSCGGLSEFIDLERYERYSGGFIWDYIDQGLVQRLPDGSERLSVGGEWGDRPTDYEFVGNGIVFADRTPSPKAQEVKQLYSPVKLAPDGHGVTIENRNLFAGTDGYVFAARLLEDGHEIWHADYRFDVAAGDTQHHDIAFPDIDADGDTREVTYEVDLLLAEATAWAPAGYELAFGQLTGTLNPEQDITETSHDDDGRATRTLSRWNAGIRRDDKEILLSRTQGGIVSWKRDDREMVIRRPELVTFRPLTDNDRGNHSGFDRAAWFAAGRYAIVTETKIHESDDGLVAEYQYELADPNHTPVSVTYHVNSDMRMQLTVEYPGNATDMASLPAFGIEWELPGEYDRLRYYGPGPEETYRDRKQGGKLGIWDATAKASMAPYLMVQETGSHEDVRWLEATDIQGHGLRVTQRGDRHFTASLLPWNTYMIEAARRHEDLPEPRHNYLRLLAAQMGVGGDDSWGAPVHTAYQLPAGRPLTLDVNLELI

SEQ ID NO: 9 - protein, 1055 aa, Bifidobacterium breve
MTNSMQGKAKTIMTNLQSAQQFSQAWLTDPRVFAVNRLAAHSSHKFYDHSPQCGEAMDLKQSLDGQWRVQMLDLADLADNELAEAAFAQPGYDAAGFSPIEVPSALETKGFLNHQYVNQQYPWSGHESPVAPDVPKHNHVALYRHEFSLEPKAAAVLEANKTAADDAAKRRVTLTFQGAATAIVVWLNGAFIGYAEDSFTPSEFDVTDVLRDGVNTLAVACFEFSSASWLEDQDFWRLHGIFRSVELEAQPLVHVNDLRVLADYDHTTGEGSLDVVALLRNAGTAAAVAATVLDAAGNTVWHSKLTAGADAETLTVKANVGKVNPWSAEEPTLYTLQVVATDAAGQVIEAALQRIGFRHFAIEDGLMKLNGKRIVFKGVDRHEFDARTGRTIAEADMIEDIHSFKRLNINAVRTSHYPNETRWYELCDEYGIYVLDETNLETHGSWTDPGDVFQPARAIPGSKDEWRAACVDRTASMVRRDYNHPSVVIWSLGNEAFGGDVFYSMRDFVHENDPFRPVHYEGTFNDPEFSAATDIMSRMYAKPDEIVKLYLGEDGKKPYISCEYSHSMGNSTGGLHLYTELERYPLYQGGFIWDYVDQALWQDCGNGTERLAYGGDFEDRPNDYEFSGDGVMFADRTPSPKAQEVKQLYANVKLVPDESGVTITNDNLFISTASSLFTARVLVDGAERWHANYRFDVPAGETVREPIAFPKVTDLVALSGSAEVTYEVDQRLAEATDWAPAGYELTFGQYVAAVSFDDGAADAVVAGDAEVAADGFNAGIHTDFGEVLLSKTQGGMVSFKRDGREMVIRRPNLTTFRALTDNDRGNGSGFERAQWMAAGRYARVTGTSVEETADGKGLKATYSYELADAKHTPVTVHYEVDAALRVHLTVEYPGEADAATLPAFGLEWILPKQYDRLRFYGLGPEETYADRLHGAKLGVFSRTAAEDCAPYLLPQETGNHEQVRWAEITDEYGHGMRVTAAGGTRFATSLLPYSSLMFEDALHQNELPKPRHTFLRLLAAQMGVGGDDTWGAPVHDEFQVPADQPLKLDVTLELI

SEQ ID NO: 10 - protein, 689 aa, Bifidobacterium catenulatum
MTQRRSYRWPQPLAGQQARIWYGGDYNPDQWPEEVWDDDVRLMKKAGVNLVSVGIFSWAKIETSEGVYDFDWLDRIIDKLGEAGIAVDLASATASPPMWLTQAHPEVLWKDYRGDVCQPGARQHWRPTSPVFREYALKLCRAMAEHYKGNPYVVAWHVSNEYGCHNRFDYSEDAERAFRKWCEERYGTIDAVNDAWGTAFWAQRMNDFTEIVPPRFIGDGNFMNPGKLLDFKRFSSDALKAFYVAERDALAEITPDLPLTTNFMVSAAGSVLDYDDWGREVDFVSNDHYFIPGEAHLDELAFSASLVDGIARKDPWFLMEHSTSAVNWRPVNYRKEPGQLVRDSLAHVAMGADAVCYFQWRQSKAGAEKFHSAMVPHTGEDSAVFRDVCELGADLNTLADNGLLGTKLAKSKVAVVFDYESEWATEHTATPTQKVHHVDEPLQWFRALADHGVTADVVPVSSNWDEYEVVVLPSVYILSEETTRRVRDYVVNGGRLIVTYYTGLSDEKDHVWLGGYPGSIRDVVGVRVEEFMPMGDDFPGVPDCLGLSNGAVAHDIADVIGSVDGTATVLETFRDDPWTGMDGAPAIVANTFGEGRSVYVGARLGRDGIAKSLPEIFESLGMAETGENDSRVLRVEREGSDGSRFVFSFNRTHEAVQIPFEGKIVVSSFAEVSGENVSIKPNGVIVTKQ

SEQ ID NO: 11 - protein, 1023 aa, Bifidobacterium catenulatum
MANSNRVEHASETWLTDATVFEVNRTPAHSNHKCFTHDPQSGEHSDLTQSLDGEWRVEIVQASDIDFNEEPFVAENFDDSSFCRAQVPGHLQMAGLLKNKYVNIQYPWDGHENPLEPNVPENNHVALYRRKFVVSKRLADTKESEGSVSIVFHGMATAIYVWVNGLFAGYGEDGFTPNEFDITDLLHDGENVVAVACYEYSSASWLEDQDFWRLHGLFRSVELTAQPHVHVENMQLEADWDAESGTASLDAALSVRNASDAATISATLKDSEGNVVWEASTNADANTTFASGSLQGLEPWSAESPSLYELEVNVIDQAGNIVEAAVQKVGFRRFRIENGIMTLNGKRIVFKGADRHEFDAKRGRSITEQDMIDDVIFCKRHNINAIRTSHYPNQERWYDLCDEYGIYLIDETNLETHGSWCLPGDVVTAETAVPGSKAHWEGACVDRVNSMVRRDYNHPSVVIWSLGNESYTGDVFRAMYKHVHDIDPNRPVHYEGVTKNRDYDDVTDIETRMYEHADVVEEYLKNDPQKPYISCEYMHAMGNSVGNLDEYTALERYPHYQGGFIWDFIDQAIYATQPDGSTRLCYGGDFGDRPSDYEFSGNGLVFADRTPTPKAQEVKQLYSNVHIDVTDRSVSIKNDNLFISTGGYQFVLRILADGEPVWQSERRFDVPADSACTFDVEWPVDLYRANADELVLEVSQRLAEATDWAPAGYELAFGQTIVAGTKAAEDAALPADGIVTVGRWNAGVQGSGREILLSRTQGGLVSYTFDGHEFVLRRPAITTFRALTDNDRGAGHGFERAQWMVAGRYARCVDNVIEQVDEDTLKAVYTYELATPQCTKVTVGYTADTTGRLNLHVEYPGESGELPTIPAFGIEWTLPVQYSNLRFFGAGPEETYQDRKHAKLGVWSTDAFKDHAPYLMPQETGNHEEVRWAEITDENGHGLRVSRANGAAPFAVSLQPYSSFMIEEAQHQDELPAPKHMFLRVLAAQMGVGGDDSWMSPVHSQYHITADQPISLDVNLELI

SEQ ID NO: 12 - protein, 1008 aa, Lactobacillus delbrueckii
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFDLTNNYTWVRALSAQMGVGGDDSWGQKVHPEFCLDAQKARQLRLVIQPLLLK

SEQ ID NO: 13 - protein, 1007 aa, Lactobacillus delbrueckii
MSNKLVKEKRVDQADLAWLTDPEVYEVNTIPPHSDHESFQSQEELEEGKSSLVQSLDGNWLIDYAENGQGPINFYAEDFDDSNFKSVKVPGNLELQGFGQPQYVNIQYPWDGSEEIFPPQVPSKNPLASYVRYFDLDEALWDKEVSLKFAGAATAIYVWLNGHFVGYGEDSFTPSEFMVTKFLKKEGNRLAVALYKYSSASWLEDQDFWRLSGLFRSVTLEAKPLLHLEDLKLTASLTDNYQKGKLEVEANIAYRLPNASFKLEVRDSEGDLVAEKVGPIRSEKLGFSLADLPVAAWSAEKPNLYQVRLYLYQAGSLLEVSRQEVGFRNFELKDGIMYLNGQRIVFKGVNRHEFDSKLGRAITEADMIWDIKTMKQSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGDFDDRPTDYEFCGDGLVFADRTTSPKLANVKALYSNLKLEVKDGQLFIKNDNLFTNSSAYYFLASLLVDGKLTYQSQPLTFGLEPGESGTFVLPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSALVKTTFTLPVALKGDLTITYEVDSLGKIAVTANFPGAVENGLLPAFGLNFALPKELSDYRYYGLGPNESYADRLEGSYLGIYQGAVEKNFTPYLRPQEAGNRSKVRYYQLFDEEGGLEFTANGADLNLSALPYSAAQIEAADHAFELTNNYTWVRALAAQMGVGGDDSWGQKVHPEFCLDAQEARQLKLVIQPLLLK

SEQ ID NO: 14 - protein, 1008 aa, Lactobacillus delbrueckii
MSNKLVKEKRVDQADLAWLTDPEVYEVNTIPPHFDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 15 - protein, 1008 aa, Lactobacillus delbrueckii
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 16 - protein, 1008 aa, Lactobacillus delbrueckii
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNIQYPWDGSEEIFPPQVPSKNPLASYVRYFDLDEAFWDKEVSLKFAGAATAIYVWLNGHFVGYGEDSFTPSEFMVTKFLKKENNRLAVALYKYSSASWLEDQDFWRLSGLFRSVTLQAKPLLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGVNRHEFDSKLGRAITEEDMIWDIKTMKRSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGGDFDDRPTDYEFCGNGLVFADRTTSPKLANVKALYSNLKLEVKDGQLFLKNDNLFTNSSAYYFLTSLLVDGKLTYQSQPLTFGLEPGESGTFVLPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSVLVKTAFTLPVALKGDLTITYEVDSLGKIAVTANFPGAVENGLLPAFGLNFALPKELSDYRYYGLGPNESYADRLEGSYLGIYQGAVEKNFTPYLRPQEAGNRSKVRYYQLFDEESGLEFTANGADLNLSALPYSAAQIEAADHAFELSNNYTWVRALAAQMGVGGDDSWGQKVHPEFCLDAQEARQLKLVIQPLLLK

SEQ ID NO: 17 - protein, 1008 aa, Lactobacillus delbrueckii
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGVNRHEFDSKLGRAITEEDMIWDIKTIKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFDLTNNYTWVRALSAQMGVGGDDSWGQKVHPEFCLDAQKARQLRLVIQPLLLK

SEQ ID NO: 18 - protein, 1008 aa, Lactobacillus delbrueckii
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIGSEQLEFTLADLPVAAWSAEKPNLYQVRLYLYQAGSLLEVSRQEVGFRNFELKDGIMYLNGQRIVFKGVNRHEFDSKLGRAITEEDMIWDIKTIKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 19 - protein, 1007 aa, Lactobacillus delbrueckii
MSNKLVKEKRVDQADLAWLTDPEVYEVNTIPPHSDHESFQSQEELEEGKSSLVQSLDGNWLIDYAENGQGPINFYAEDFDDSNFKSVKVPGNLELQGFGQPQYVNIQYPWDGSEEIFPPQVPSKNPLASYVRYFDLDEALWDKEVSLKFAGAATAIYVWLNGHFVGYGEDSFTPSEFMVTKFLKKEGNRLAVALYKYSSASWLEDQDFWRLSGLFRSVTLEAKPLLHLEDLKLTASLTDNYQKGKLEVEANIAYRLPNASFKLEVRDSEGDLVAEKVGPIRSEKLDFSLADLPVAAWSAEKPNLYQVRLYLYQAGSLLEVSRQEVGFRNFELKDGIMYLNGQRIVFKGVNRHEFDSKLGRAITEADMIWDIKTMKQSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGDFDDRPTDYEFCGDGLVFADRTTSPKLANVKALYSNLKLEVKDGQLFIKNDNLFTNSSAYYFLTSLLVDGKLTYQSQPLTFGLEPGESGTFALPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSALVKTAFTLPVALKGDLTITYEVDSLGKIAVTANFPGAVENGLLPAFGLNFALPKELSDYRYYGLGPNESYADRLEGSYLGIYQGMVEKNFTPYLRPQEAGNRSKVRYYQLFDEEGGLEFTANGADLNLSALPYSAAQIEAADHAFELTNNYTWVRALAAQMGVGGDDSWGQKVHPEFCLDAQEARQLKLVIQPLLLK

SEQ ID NO: 20 - protein, 628 aa, Lactobacillus helveticus
MQANINWLDNPEVFRVNQLPAHSDHPFFRDYREWQKQHSSYQQSLNGKWKFHFSANPMDRPQDFYQRDFDSSNFDSIPVPSEIELSNYTQNQYINVLFPWEGKIFRRPAYALDPNDHEEGSFSKGADNTVGSYLKRFDLSSALIGKDVHIKFEGVEQAMYVWLNGHFVGYAEDSFTPSEFDLTPYIQDKDNLLAVEVFKHSTASWLEDQDMFRFSGIFRSVELLGIPATHLMDMDLKPRVADNYQDGIFNLKLHFIGKKAGSFHLLVKDIKGHTLLEKNEDIKENVQINNEKFENVHLWNNHDPYLYQLLIEVYDEQQNLLELIPFQFGFRRIEISPEKVVLLNGKRLIINGVNRHEWDAKRGRSITMSDMTTDINTFKENNINAVRTCHYPNQIPWYYLCDQNGIYVMAENNLESHGTWQKMGEIEPSDNVPGSIPQWKEAVIDRARNNYETFKNHTSILFWSLGNESYAGDNIIAMNEFYKSHDDTRLVHYEGVVHRPELKDKISDVESCMYLPPKKVEEYLQNDPPKPFMECEYMHDMGNSDGGMGSYIKLLDKYPQYFGGFIWDFIDQALLVHDEISGHDVLRYGGDFDDRHSDYEFSGDGLMFADRTPKPAMQEVRYYYGLHK

SEQ ID NO: 21 - protein, 318 aa, Lactobacillus helveticus
MDYTNNQLHIIYGDATFGVNGKDFQYIFSYERGGLESLKVHGKEWLYRVPTPTFWRATTDNDRGSGFNLKAAQWLGADMFTKCTDIHLKVDRHDFAELPIAPFNNKFSNHEYAKSAEISFTYQTLTTPATNAKIIYNIDDVGHIKVTMRYYGKKGLPPLPVIGIRLIMPTAATGFDYEGLSGETYPDRMAGAKEGKFHIDGLPVTEYLVPQENGMHMQTKKLTINRETTQNNVDRTNEKFSLSIQQAEKPFNFSCLPYTAEELENATHIEELPLVRRTVLVIAGAVRGVGGIDSWGTDVESAYHINPELDHEFSFILN

SEQ ID NO: 22 - protein, 1023 aa, Bifidobacterium longum
MTDVTHVDRASQAWLTDPTVFEVNRTPAHSSHKWYARDPQSGQWSDLKQSLDGEWRVEVVQAADINLEEEPATAESFDDSSFERIQVPGHLQTAGLMNHKYVNVQYPWDGHENPLEPNIPENNHVALYRRKFTVSAPVANAKQAGGSVSIVFHGMATAIYVWVNGAFVGYGEDGFTPNEFDITELLHDGENVVAVACYEYSSASWLEDQDFWRLHGLFRSVELAARPHVHIENTQIEADWDPEAGTASLDAALTVLNAADAATVRATLKDADGNTVWQTTGDAEAQTAISSGPLQGIAPWSAESPTLYELDVDVIDQAGDVIECTSQKVGFRRFRIEDGILTINGKRIVFKGADRHEFDAEQGRAITEQDMIDDVVFCKRHNINSIRTSHYPNQERWYELCDEYGIYLIDEANLEAHGSWSLPGDVLTEDTIVPGSKREWEGACVDRVNSMMRRDYNHPSVLIWSLGNESYVGDVFRAMYKHVHDIDPNRPVHYEGVTHNRDYDDVTDIETRMYSHADEIEKYLKDDPKKPYLSCEYMHAMGNSVGNMDEYTALERYPKYQGGFIWDFIDQAIYATQPDGTRSLRYGGDFGDRPSDYEFSGDGLLFANRKPSPKAQEVKQLYSNVHIDVTKDSVSVKNDNLFTATGDYVFVLSVLADGKPVWQSTRRFDVPAGETRTFDVAWPVAAYRADARELVLQVSQRLAKATDWAESGYELAFGQTVVPADATATPDTKPADGTITVGRWNAGVRGAGREVLLSRTQGGMVSYTFAGNEFVLRRPAITTFRPLTDNDRGAGHGFERVQWLGAGRYARCVDNVLEQIDDSTLKGTYTYELATAQRTKVTVSYTAHTDGRVNLHVEYPGEQGDLPTIPAFGIEWTLPVQYTNLRFFGTGPAETYLDRKHAKLGVWSTNAFADHAPYLMPQETGNHEDVRWAEITDDHGHGMRVSRADGAAPFAVSLLPYSSFMLEEAQHQDELPKPKHMFLRVLAAQMGVGGDDSWMSPVHPQYHIPADKPISLDVDLELI

SEQ ID NO: 23 - protein, 628 aa, Lactobacillus reuteri
MDADIKWLDEPETFRVNQLPAHSDHYYYGNYDEWRHNNSRFAQNLDGQWQFNFAENLRERENDFYKMDYDSSSFGTIEVPSEIELNNYAQNNYINTLIPWEGKIYRRPAYTLSPDDAQEGSFSDGDDNTIGEYLKHFDLDPSLRGKQVRIRFDGVERAMYVWLNGHFIGYAEDSFTPSEFDLTPYIQDEGNVLAVEVFKHSTASWIEDQDMFRFSGIFRSVNLLAQPLVHVEDLNIRPIVTDNYQDGIFNVELQLHGEKTGNVNVRVIDNDGNTLVNETHPVDSTVKVQDQFLENVHLWDNHDPYLYQLLIEIRDDEGNLVELVPYRFGFRRIEINKDHVVLLNGQRLIINGVNRHEWDARRGRAITMDDMTSDIHTFKENNINAVRTCHYPDQIPWYYLCDDNGIYMMAENNLESHATWQKMGAIEPSYNVPGSVPQWRDVVVDRARTNYETFKNHPSILFWSLGNESYAGDNIVKMNEFYKKHDDSRLVHYEGVCHTPEYRDRISDVESWMYLPPKEVEEYLKNNPDKPFMECEYMHDMGNSDGGMGSYISLLDKYPQYFGGFIWDFIDQALLVKDPVSGQEVMRYGGDFDDRHSDYEFSGDGLMFADRTPKPAMQEVRYYYGLHK

SEQ ID NO: 24 - protein, 319 aa, Lactobacillus reuteri
MAYTNKLRVIYGDATLGLSGDGFHYIFSYERGGLESLKLNGKEWLYREPMPTFWRATTDNDRGSGFNIRSAQWLAADTFHKCVGIDLTVDNQHFAELPIAPITNEFSDPVSAESVKIKYTFATLTVPATQVTVIYEVNGQGEIKVTMHYYGHEDLPGLPVVGMRFIMPTVATGFDYQGLSGETYPDRMAGATEGTFHVDGLPVTKYLVPQENGMHMATHALTITRDSTQNNADHSREPFSLTVKQDAQPFAFSCLPYTAEELENATHIEELPLARRTVLVVAGAVRGVGGIDSWGADVEEQYHIPADRDVEFSFVLNAK

SEQ ID NO: 25 - protein, 1007 aa, Lactobacillus delbrueckii
MSNKLVKEKRVDQADLAWLTDPEVYEVNTIPPHSDHESFQSQEELEEGKSSLVQSLDGNWLIDYAENGQGPINFYAEDFDDSNFKSVKVPGNLELQGFGQPQYVNIQYPWDGSEEIFPPQVPSKIPLASYVRYFDLDEALWDKEVSLKFAGAATAIYVWLNGHFVGYGEDSFTPSEFMVTKFLKKEGNRLAVALYKYSSASWLEDQDFWRLSGLFRSVTLEAKPLLHLEDLKLTASLTDNYQKGKLEVEANIAYRLPNASFKLEVRDSEGDLVAEKVGPIRSEKLDFSLADLPVAAWSAEKPNLYQVRLYLYQAGSLLEVSRQEVGFRNFELKDGIMYLNGQRIVFKGVNRHEFDSKLGRAITEADMIWDIKTMKQSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGDFDDRPTDYEFCGDGLVFADRTTSPKLANVKALYSNLKLEVKDGQLFIKNDNLFTNSSAYYFLTSLLVDGKLTYQSQPLTFGLEPGESGTFALPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSALVKTTFTLPVALKGDLTITYEVDSLGKIAVTANFPGAVENGLLPAFGLNFALPKELSDYRYYGLGPNESYADRLEGSYLGIYQGMVEKNFTPYLRPQEAGNRSKVRYYQLFDEEGGLEFTANGADLNLSALPYSAAQIEAADHAFELTNNYTWVRALAAQMGVGGDDSWGQKVHPEFCLDAQEARQLKLVIQPLLLK

SEQ ID NO: 26 - protein, 628 aa, Lactobacillus helveticus
MQANINWLDNPEVFRVNQLPAHSDHPFFRDYREWQKQHSSYQQSLNGKWKFHFSANPMDRPQDFYQRDFDSSNFDSIPVPSEIELSNYTQNQYINVLFPWEGKIFRRPAYALDPNDHEEGSFSKGADNTVGSYLKRFDLSSALIGKDVHIKFEGVEQAMYVWLNGHFVGYAEDSFTPSEFDLTPYIQEKDNLLAVEVFKHSTASWLEDQDMFRFSGIFRSVELLGIPATHLMDMDLKPRVADNYQDGIFNLKLHFIGKKAGSFHLLVKDIKGHTLLEKNEDIKENVQINNEKFENVHLWNNHDPYLYQLLIEVYDEQQNLLELIPFQFGFRRIEISPEKVVLLNGKRLIINGVNRHEWDAKRGRSITMSDMTTDINTFKENNINAVRTCHYPNQIPWYYLCDQNGIYVMAENNLESHGTWQKMGEIEPSDNVPGSIPQWKEAVIDRARNNYETFKNHTSILFWSLGNESYAGDNIIAMNEFYKSHDDTRLVHYEGVVHRPELKDKISDVESCMYLPPKKVEEYLQNDPPKPFMECEYMHDMGNSNGGMDSYIKLLDKYPQYFGGFIWDFIDQALLVHDEISGHDVLRYGGDFDDRHSDYEFSGDGLMFADRKPKPAMQEVRYYYGLHK

SEQ ID NO: 27 - protein, 318 aa, Lactobacillus helveticus
MDYTNNQLHIIYGDATFGVNGKDFQYIFSYERGGLESLKVHGKEWLYRVPTPTFWRATTDNDRGSGFNLKAAQWLGADMFTKCTDIHLKVDRHDFAELPIAPFNNKFSNHEYAKSAEISFTYQTLTTPATNAKIIYNIDDGGHIKVTMRYYGKKGLPPLPVIGIRLIMPTAATGFDYEGLSGETYPDRMAGAKEGKFHIDGLPVTEYLVPQENGMHMQTKKLTINRETTQNNVDRTNEKFSLSIQQAEKPFNFSCLPYTAEELENATHIEELPLVRRTVLVIAGAVRGVGGIDSWGTDVESAYHINPDLDHEFSFILN

SEQ ID NO: 28 - protein, 626 aa, Lactobacillus crispatus
MKANIKWLDDPEVFRINQLPAHSDHPFYKDYREWQKHSSSFKQSLNGAWQFHFSKDPQSRPIDFYKLSFDSSSFDTIPVPSEIELNGYAQNQYTNILYPWESKIYRKPAYTLGRGIKDGDFSQGKDNTVGSYLKHFDLNPALAGHDIHIQFEGVERAMYVYLNGHFIGYAEDSFTPSEFDLTPYIQAKDNILAVEVFKHSTASWLEDQDMFRFSGIFRSVELLALPRTHLMDLDIKPTVVNDYHDGVFNAKLHFMGKTSGNVHVLIEDIDGKTLLNKKLPLKSTVEIENETFANVHLWDNHDPYLYQLIIEVHDQDGKLVELIPYQFGFRKIEITKDHVVLLNGKRLIINGVNRHEWDAKRGRSITLADMKQDIATFKHNNINAVRTCHYPNQIPWYYLCDQNGIYMMAENNLESHGTWQKLGQVEATSNVPGSIPEWREVVVDRARSNYETFKNHTAILFWSLGNESYAGSNIAAMNKLYKDHDSSRLTHYEGVFHAPEFKKEISDLESCMYLPPKEAEEYLQNPKKPLVECEYMHDMGNSDGGIGSYIKLIDKYPQYMGGFIWDFIDQALLVHDPVSGQDVLRYGGDFDDRHSDYEFSGDGLMFADRTPKPAMQEVRYYYGLHK

SEQ ID NO: 29 - protein, 316 aa, Lactobacillus crispatus
MAYTNNLHVVYGEASLGVNGQDFAYLFSYERGVLESLKIKDKEWLYRTPTPTFWRATTDNDRGSGFNQKAAQWLGADMFTKCVGIHVQVDDHQFDELPIAPINNQFSNQEFAHEVKVAFDYETLTTPATKVKIIYNINDAGHMTITMHYFGKKGLPPLPVIGMRFIMPTKAKSFDYTGLSGETYPDRMAGAERGTFHIDGLPVTKYLVPQENGMHMQTNELVITRNSTQNNADKDGDFSLKITQTKQPFNFSLLPYTAEELENATHIEELPLARRSVLVIAGAVRGVGGIDSWGSDVEEQYHIDPEQDHEFSFTLN

SEQ ID NO: 30 - protein, 1025 aa, Streptococcus thermophilus
MNMTKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATKWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDSWGAPVHDEFLLSSADSYQLSFMIEPLN

SEQ ID NO: 31 - protein, 1008 aa, Lactobacillus delbrueckii
MNNKLAQVKRVDQADLAWLTDPEIYEVNTIPPHSDHESFQSLEELEEGKSSLVQSLDGDWLIDYAENGEGPANFYEENFDDSSFKSVKVPGNLELQGFGQPQYVNVQYPWDGSDEIFPPMIPSKNPVASYVRYFDLEEAFWDKEVSLKFAGAATAIYVWLNGHFVGYGEDSFTPSEFMVTKFLKKEGNRLAVALYKYSSASWLEDQDFWRMSGLFRSVTLEAKPLLHLQDLKLTASLTNDYQKGSLQVEADIDYRLPNSSFKLELRDSAGELVAEKVGPIRSEKLDFSLADLPVAAWSAEEPNLYQVRLSLYQQGSLLEVSRQEVGFRNFELKDGIMYLNGKRIVFKGVNRHEFDSKLGRAITEADMIWDIKTMKQSNINAVRCSHYPNQSIFYHLCDKYGLYVIDEANLESHGTWEKVGGHEDPSFNVPGDDQRWLGASLSRVKNMMARDKNHASILIWSLGNESYAGKVFAQMADYVRQADPTRVQHYEGVTHNRKFDDATQIESRMYAPAKEIEEYLTKKPAKPFVSCEYAHAMGNSVGDLAAYTALEKYPHYQGGFIWDWIDQGLEKEGHLLYGGDFDDRPSDYEFCGDGLVFADRTTSPKLANVKALYSNLKLELKDGQLFLKNDNLFTNSSAYYFLTSLLVDGKLTYQSQPLTFALEPGESGTFALPWPEVEDEKGEIVYQVTAHLKEDLPWADEGFTVAEAEEAVTKLPEFYPAGRPELVDSDYNLGIKGNGFRILFSKAKGWPVSIKYAGREYLKRLPEFTFWRALTDNDRGAGYGYDLAKWENAGKYARLQDISYEIKENSVLVKTAFTLPVALKGDLTITYEVDSLGKIAVTANFPGAVENGLLPAFGLNFALPKELSDYRYYGLGPNESYADRLEGSYLGIYQGAVEKNFTPYLRPQEVGNRSKVRYYQLFDEEGGLEFTANGANLNLSALPYSAAQIEAADHAFELTNNYTWVRALAAQMGVGGDDSWGQKVHPEFCLDAQEARQLKLVIQPLFTE

SEQ ID NO: 32 - protein, 1049 aa, Bifidobacterium adolescentis
MADTAELAIVHATTASASWLTDPTVFAANRKPAHSSHRYVIGETSEPKQSLDGEWKVRIEQARNVDVESAPFAAVDFEDGDFGAIEVPGHLQMAGYLKNKYVNIQYPWDGHEDPQAPNIPENNHVAIYRRRFALDAQLARTLENDGTVSLTFHGAATAIYVWLDGTFVGYGEDGFTPSEFDVTEALRNGNGNAADSPEAEHTLTVACYEYSSASWLEDQDFWRLHGLFRTVELAAQPHTHVETVQLEADYTAADTAGTADTAELNAALTLRNPADAMTIESTLRDGDGNVVWESTQACNGEIALNSGKMTNIAPWSAESPTLYTLTVRVVGHDGAIIETVTQKIGFRTFRIENGIMTLNGKRIVFKGADRHEFDAKRGRAITREDMLSDVVFCKRHNINAIRTSHYPNQEYWYDLCDEYGLYLIDETNMETHGTWVANNVERPEDGIPGSRPEWEDACVDRINSMMRRDYNHPSVLIWSLGNESSAGEVFRAMYRHAHTIDPNRPVHYEGSVHMREFEDVTDIESRMYAHADEIERYLNDGSPAHTDGPKKPYISCEYMHAMGNSCGNMDEYTALERYPMYQGGFIWDFIDQAIETKLPDGTTRMCYGGDFGDRPSDYEFSGDGLLFADRTPSPKAQEVKQLYANVKIAVSVDEARITNDNLFVSTGDYRFVLRILADGKPVWSTTRRFDVAAGESASFEVDWPVDDYRSNAEELVLEVSQQLGNACDWAPAGYELAFGQCVVAGAKTTADAVDAAGAPADGTVTLGRWNAGVRGQGREALFSRTQGGMVSYTFGEREFVLRRPSITTFRPLTDNDRGAGHAFERAAWAVAGKYARCVDCAIANRGENAVEATYTYELAIPQRTKVTVRYVADTAGLVSLDVEYPGEKNGDLPTIPAFGIEWALPVEYANLRFYGAGPEETYADRRHAKLGVWSTTAGDDCAPYLLPQETGNHEDVRWAEITDDSGHGVRVKRGAGAKPFAMSLLPYSSTMLEEALHQDELPKPRHMFLRLLAAQMGVGGDDSWMSPVHEQYQLPADQPLSLNVQLKLF

SEQ ID NO: 33 - protein, 1023 aa, Bifidobacterium adolescentis
MANETRIEHASETWLADSTVFEVNRVPAHSDHKCYAHDSQTNEWSDLRQSLDGEWRVEVVQASDIEFNEEPFVRENFDDSAFERIQVPGHLQMAGLMNNKYVNIQYPWDGHENPAEPNIPENNHVALYRKTFTMANRLADTKNAGGTVSIVFHGMATAIYVWVNGMFVGYGEDGFTPNEFDITEMLHDGENVVAVACYEYSSASWLEDQDFWRLHGLFRSVELAAQPHVHIENMQIESDWDPESGSASLDAALTVRNAADAATISATLKDSDGNVVWETANCADPDTSISTGSLNGIRPWSAEDPVLYEFEVTVIDHAGNIAEVAVQKVGFRRFRIEDGIMTINGKRIVFKGADRHEFDPKRGRAITEQDMIDDVVFCKRHNLNAIRTSHYPNQERWYELCDEYGIYLIDETNLETHGSWCLPGDVLTEETAVPGSKAHWEGACVDRVNSMVRRDYNHPSVLIWSLGNESYTGDVFRAMYKRVHDIDPNRPVHYEGVTHNRDYNDVTDIETRMYAHADAIEEYLKNDPQKPYISCEYMHAMGNSCGNMDEYTALERYPKYQGGFIWDFIDQAIYATQPDGTTSLRYGGDFGDRPSDYEFSGNGLVFADRKPTPKAQEVKQLYSNVHIDVAEDSVTIKNDNLFTSTGEYTFVLRVLADGEPVWQSERRFDVPAGSTEKLDVDWPLDLYRDGASELVLEVSQRLAKATNWAVAGYELAFGQTVVAGSKKASAPVKPVDGIVTVGRWNVGVQGSGREVLLSRTQGGLVSYTFNNREFVLRRPAVTTFRALTDNDRGAGHGFERAQWLGAGRYARCIGNEIEQIDENTVKASYTYELATPQRTKVTVSYTADTTGRVNLHVEYPGEPGDLPTIPAFGIEWTLPVQYSNLRFFGAGPEETYQDRKHAKLGVWSTDAFKDHAPYLMPQETGNHEDVRWAEITDEKGHGLRISRAEGAEPFAMSLQPYSSFMLEEAQHQDELPAPKHMFLRVLAEQMGVGGDDSWMSPVHPQYHIPADQPISLDVDLDLI

SEQ ID NO: 34 - protein, 1305 aa, Bifidobacterium bifidum
MVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTEAAANITLKQTVFPKGGKTDAAIGTVTTASKSIAAGASADVTSTITAASPKLWSIKNPNLYTVRTEVLNGDTVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQTIAGDNAVLGGDKDETWAKFDLTSTINRDRNAPSVIMWSLGNEMMEGISGSVSDFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNKVPVVVYTDAAKVKLYFTPKGSTEKRLIGEKSFTKKTTAAGYTYQVYEGTDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERD

SEQ ID NO: 35 - protein, 1025 aa, unidentified
  source 1..1025: note = Kluyveromyces lactis
MSCLIPENLRNPKKVHENRLPTRAYYYDQDIFESLNGPWAFALFDAPLDAPDAKNLDWETAKKWSTISVPSHWELQEDWKYGKPIYTNVQYPIPIDIPNPPTVNPTGVYARTFELDSKSIESFEHRLRFEGVDNCYELYVNGQYVGFNKGSRNGAEFDIQKYVSEGENLVVVKVFKWSDSTYIEDQDQWWLSGIYRDVSLLKLPKKAHIEDVRVTTTFVDSQYQDAELSVKVDVQGSSYDHINFTLYEPEDGSKVYDASSLLNEENGNTTFSTKEFISFSTKKNEETAFKINVKAPEHWTAENPTLYKYQLDLIGSDGSVIQSIKHHVGFRQVELKDGNITVNGKDILFRGVNRHDHHPRFGRAVPLDFVVRDLILMKKFNINAVRNSHYPNHPKVYDLFDKLGFWVIDEADLETHGVQEPFNRHTNLEAEYPDTKNKLYDVNAHYLSDNPEYEVAYLDRASQLVLRDVNHPSIIIWSLGNEACYGRNHKAMYKLIKQLDPTRLVHYEGDLNALSADIFSFMYPTFEIMERWRKNHTDENGKFEKPLILCEYGHAMGNGPGSLKEYQELFYKEKFYQGGFIWEWANHGIEFEDVSTADGKLHKAYAYGGDFKEEVHDGVFIMDGLCNSEHNPTPGLVEYKKVIEPVHIKIAHGSVTITNKHDFITTDHLLFIDKDTGKTIDVPSLKPEESVTIPSDTTYVVAVLKDDAGVLKAGHEIAWGQAELPLKVPDFVTETAEKAAKINDGKRYVSVESSGLHFILDKLLGKIESLKVKGKEISSKFEGSSITFWRPPTNNDEPRDFKNWKKYNIDLMKQNIHGVSVEKGSNGSLAVVTVNSRISPVVFYYGFETVQKYTIFANKINLNTSMKLTGEYQPPDFPRVGYEFWLGDSYESFEWLGRGPGESYPDKKESQRFGLYDSKDVEEFVYDYPQENGNHTDTHFLNIKFEGAGKLSIFQKEKPFNFKISDEYGVDEAAHACDVKRYGRHYLRLDHAIHGVGSEACGPAVLDQYRLKAQDFNFEFDLAFE

SEQ ID NO: 36 - DNA, 30 bp, synthetic construct
  misc_feature 1..30: note = Description of Artificial Sequence: Synthetic primer
  modified_base 9: mod_base = OTHER; note = deoxyuracil
  source 1..30: mol_type = other DNA
attaaccatgcgacgcaacttcgaatggcc

SEQ ID NO: 37 - DNA, 31 bp, synthetic construct
  misc_feature 1..31: note = Description of Artificial Sequence: Synthetic primer
  modified_base 9: mod_base = OTHER; note = deoxyuracil
  source 1..31: mol_type = other DNA
atcttctctttaccgccttaccacgagcacg

SEQ ID NO: 38 - DNA, 33 bp, synthetic construct
  misc_feature 1..33: note = Description of Artificial Sequence: Synthetic primer
  modified_base 9: mod_base = OTHER; note = deoxyuracil
  source 1..33: mol_type = other DNA
agagaagattttcagcctgatacagattaaatc

SEQ ID NO: 39 - DNA, 33 bp, synthetic construct
  misc_feature 1..33: note = Description of Artificial Sequence: Synthetic primer
  modified_base 9: mod_base = OTHER; note = deoxyuracil
  source 1..33: mol_type = other DNA
atggttaattcctcctgttagcccaaaaaacgg

SEQ ID NO: 40 - DNA, 22 bp, synthetic construct
  misc_feature 1..22: note = Description of Artificial Sequence: Synthetic primer
  source 1..22: mol_type = other DNA
cggcgtcacactttgctatgcc

SEQ ID NO: 41 - DNA, 20 bp, synthetic construct
  misc_feature 1..20: note = Description of Artificial Sequence: Synthetic primer
  source 1..20: mol_type = other DNA
ccgcgctactgccgccaggc

(19) United States
(12) Patent Application Publication
(10) Pub. No.: US 2025/0324990 A1
(43) Pub. Date: Oct. 23, 2025

(54) LACTASE ENZYMES WITH IMPROVED ACTIVITY AT LOW TEMPERATURES

(71) Applicant: Kerry Group Services International Ltd, Tralee (IE)

(72) Inventors: Hans Raj, Hoersholm (DK); Pernille Smith, Broenshoej (DK); Thomas Eckhardt, Birkeroed (DK); Vojislav Vojinovic, Graested (DK); Charlotte Elisabeth Grüner Schöller, Virum (DK); Johannes Maarten van den Brink, Herlev (DK)

(73) Assignee: Kerry Group Services International Ltd, Tralee (IE)

(21) Appl. No.: 19/248,775

(22) Filed: Jun. 25, 2025

Related U.S. Application Data
(63) Continuation of application No. 16/604,134, filed on Oct. 9, 2019, now Pat. No. 12,369,597.

(30) Foreign Application Priority Data
Apr. 11, 2017 (EP) 17166021.0
Aug. 31, 2017 (EP) 17188732.6

(51) Int. Cl. A23C 9/12; C12N 9/38

(52) U.S. Cl. CPC A23C 9/1206; C12N 9/2471

(57) ABSTRACT

The present invention relates to new improved peptide or dimeric peptides exhibiting beta-galactosidase enzyme activity as well as improved methods for reducing the lactose content in compositions in particular at low temperatures.

### CROSS-REFERENCE TO RELATED APPLICATIONS

The present application is a continuation of U.S. application Ser. No. 16/604,134, which is the U.S. National Stage of International Application No. PCT/EP2018/059289, filed Apr. 11, 2018, and claims priority to European Patent Application No. 17166021.0, filed Apr. 11, 2017, and European Patent Application No. 17188732.6, filed Aug. 31, 2017.

### SEQUENCE LISTING

The instant application contains a Sequence Listing which has been filed electronically in XML format and is hereby incorporated by reference in its entirety. Said XML copy, created on Jun. 23, 2025, is named 140776-0111_SL.xml and is 70,412 bytes in size.

### FIELD OF THE INVENTION

The present invention relates to methods for producing a dairy product and methods for reducing the lactose content of a dairy product using new peptides or dimeric peptides exhibiting beta-galactosidase enzyme activity with improved activity at low temperatures.

### BACKGROUND OF THE INVENTION

In order to grow on milk, lactose hydrolysis is a good way for lactic acid bacteria to obtain glucose and galactose as carbon source. Lactase (beta-galactosidase; EC 3.2.1.23) is the enzyme that performs the hydrolysis step of the milk sugar lactose into monosaccharides. The commercial use of lactase is to break down lactose in dairy products. Lactose intolerant people have difficulties to digest dairy products with high lactose levels. It is estimated that about 70% of the world's population has a limited ability to digest lactose. Accordingly, there is a growing demand for dairy food products that contain no or only low levels of lactose.

Lactases have been isolated from a large variety of organisms, including microorganisms like Kluyveromyces and Bacillus. Kluyveromyces, especially K. fragilis and K. lactis, and other fungi such as those of the genera Candida, Torula and Torulopsis, are a common source of fungal lactases, whereas B. coagulans and B. circulans are well known sources for bacterial lactases. Several commercial lactase preparations derived from these organisms are available such as Lactozym® (available from Novozymes, Denmark), HA-Lactase (available from Chr. Hansen,

Denmark) and Maxilact® (available from DSM, the Netherlands), all from K. lactis. All these lactases are so-called neutral lactases having a pH optimum between pH 6 and pH 8, as well as a temperature optimum around 37° C. When such lactases are used in the production of, e.g. low-lactose yoghurt, the enzyme treatment will either have to be done in a separate step before fermentation or rather high enzyme dosages have to be used because their activity will drop as the pH decreases during fermentation.

A typical process for production of pasteurized milk with reduced lactose comprises addition of the lactase enzyme to the milk followed by prolonged incubation (10-48 h, often 24 h) at temperatures around 6° C. Because the Ha-Lactase and NOLA® Fit activity is in the range of 45-70 μmol per min per mg of enzyme, enzyme doses in the range of 55-70 mg/L and 45-60 mg/L respectively for pasteurized milk are required to achieve the desired residual lactose level. The Ha-Lactase and NOLA® Fit enzymes have temperature optimum around 37° C. Longer incubation of milk at 37° C. can result in microbial growth.

Also, these lactases are not suitable for hydrolysis of lactose in milk performed at high or low temperatures, which would in some cases be beneficial in order to keep the microbial count low and thus ensure high milk quality. Furthermore, the known lactases would not be suitable for use in a desired process for the production of ultra-heat treated (UHT) milk, wherein enzymes were added prior to the UHT treatment.

WO92/13068 relates to compositions comprising lactase activity obtained from sonication of microbial cells of bacteria or yeast. WO2010092057 and WO0104276 relate to cold-active beta-galactosidases. WO07110619 relates to beta-galactosidase with high transgalactosylating activity, whereas WO2009071539 relates to beta-galactosidase with lower transgalactosylating activity.

### OBJECT OF THE INVENTION

It is an object of embodiments of the invention to provide methods using beta-galactosidases that enable the production of improved lactose-free or low-lactose products at low temperatures.

It is a further object of embodiments of the invention to provide methods using beta-galactosidases with properties that improve the lowering of lactose in a product, such as lactose-free or low-lactose products.

### SUMMARY OF THE INVENTION

The present inventor(s) have identified beta-galactosidases with properties not previously described that enable the production of improved lactose-free or low-lactose products as well as enabling improved production methods for such lactose-free or low-lactose products. In particular these beta-galactosidases have been shown to be very stable with relatively high activity at a very broad range of both temperatures as well as pH values. They are also useable at specific temperatures, such as at high temperatures and pH values not normally seen with these enzymes. First of all, this enables to the use of beta-galactosidases at specific pH values and temperatures that were not known to be possible. It also enables the use of the same specific enzyme in several different applications, which is highly requested in the industry.

In a first aspect the present invention provides methods for producing a dairy product comprising:
(a) mixing a milk-based substrate comprising lactose in a concentration of at least 10 g/L and a peptide or a dimeric peptide exhibiting beta-galactosidase activity in a concentration of 10 to 55 mg/L, such as e.g. 20 to 55 mg/L;
(b) incubating the mixture at a temperature from 1° C.-10° C for a period of time sufficient to reduce the lactose concentration in the mixture to less than 0.2 g/L.

In a related embodiment the present invention provides methods for reducing the lactose content in a milk-based substrate comprising:
(a) mixing a milk-based substrate comprising lactose in a concentration of at least 10 g/L and a peptide or a dimeric peptide exhibiting beta-galactosidase activity in a concentration of 10 to 55 mg/L, such as e.g. 20 to 55 mg/L;
(b) incubating the mixture at a temperature from 1° C.-10° C. for a period of time sufficient to reduce the lactose concentration in the mixture to less than 0.2 g/L.

The methods as described above can be carried out with a peptide or dimeric peptide exhibiting beta-galactosidase activity which may be further be characterized as:
(i) a peptide having an amino acid sequence selected from SEQ ID NO: 22, 33, 14, 7, 9, 11, 30 and 1 or a peptide having an amino acid sequence identity of more than 85% to any of these sequences;
(ii) a peptide having an amino acid sequence selected from SEQ ID NO: 22, 33, 14, 13, 19, 7, 9, 11, 26 and 27, 30 and 1 or a peptide having an amino acid sequence identity of more than 85% to any of these sequences;
(iii) a peptide having an amino acid sequence represented by SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or enzymatically active fragments thereof, or an amino acid sequence of any one thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.

The methods of the present invention are advantageous as they only require a low concentration of the peptide or dimeric peptide exhibiting beta-galactosidase activity and still significantly reduce the lactose concentration. In a preferred alternative, the peptide or dimeric peptide exhibiting beta-galactosidase activity is added in a concentration of 35 to 52 mg/L, in a concentration of 40 to 52 mg/L or in a concentration of 45 to 52 mg/L.

The milk-based substrate can be any substrate containing milk. In one aspect the above methods use a milk-based substrate which is:
(i) cow milk, sheep milk, goat milk, buffalo milk, camel milk, or a pasteurized and/or filtered form thereof; or
(ii) a fermented dairy product obtained from (i) by fermentation.

In a particularly preferred embodiment, the above methods use cow milk comprising lactose in a concentration of about 37 to 50 g/L or a heat treated, pasteurized, raw and/or filtered form thereof as the milk-based substrate.

The above methods provide for a significant reduction of the concentration of lactose in a short period of time. In certain embodiments, the concentration is reduced to a value of less than 0.2 g/l lactose after incubation for at least 4 hours, at least 8 hours, at least 12 hours or at least 24 hours.

One of the advantages of the methods of the present invention resides in reduction of the concentration of lactose at low temperatures. For example the incubation temperature in step (b) of the above methods can be in the range of from 2° C.-7° C. or in the range of from 3° C.-6° C.

The methods provide a significant reduction of the concentration of lactose and preferably the incubation in step (b) reduces the lactose concentration in the mixture to less than 0.05 g/L, to less than 0.02 g/L, or to less than 0.01 g/L.

Specific the peptide or dimeric peptide exhibiting beta-galactosidase activity to be used in the methods of the invention are not only highly active at low temperatures, but also at high temperatures. In one aspect the invention thus provides method as described above, wherein the mixture comprising the milk-based substrate and the peptide or dimeric peptide exhibiting beta-galactosidase activity is heated to a temperature of at least 60° C. for at least four seconds before or after incubating the mixture at a temperature from 1° C.-10° C. In particular, the method may comprise a heating step including heating to a temperature of 72° C. for about 15 seconds before or after incubating the mixture at low temperatures in step (b) or heated to a temperature of 140° C. for about four seconds before or after incubating the mixture at a temperature from 1° C.-10° C.

In one alternative, the methods of the present invention are used for producing a dairy product. These methods may further comprise a step of fermenting the milk-based substrate with lactic acid bacteria. The fermentation step is carried out before or after the incubation with a peptide or dimeric peptide exhibiting beta-galactosidase activity.

The methods are particularly suitable for producing dairy products, such as a fermented milk product, cheese, yoghurt, butter, dairy spread, butter milk, acidified milk drink, sour cream, whey based drink, ice cream, condensed milk, dulce de leche or a flavored milk drink.

In a particularly preferred embodiment the present invention provides methods for producing milk or a dairy product comprising:
(a) mixing a milk-based substrate comprising lactose in a concentration of at least 10 g/L and a peptide exhibiting beta-galactosidase activity in a concentration of 35 to 52 mg/L, wherein the peptide has an amino acid sequence selected from SEQ ID NO: 22, 33, 14, 7, 9, 11, 30 and 1 or an amino acid sequence identity of more than 85% to any of these sequences;
(b) incubating the mixture at a temperature from 1° C.-10° C. for 12 hours under conditions sufficient to reduce the lactose concentration in the mixture to less than 0.02 g/L.

In a further preferred embodiment the present invention provides methods for producing milk or a dairy product comprising:
(a) mixing a milk-based substrate comprising lactose in a concentration of at least 10 g/L and a peptide exhibiting beta-galactosidase activity in a concentration of 35 to 52 mg/L, wherein the peptide has an amino acid sequence selected from SEQ ID NO: 22, 33, 14, 7, 9, 11, 30 and 1 or an amino acid sequence identity of more than 85% to any of these sequences;
(b) incubating the mixture at a temperature from 1° C.-10° C. for 12 hours under conditions sufficient to reduce the lactose concentration in the mixture to less than 0.02 g/L, wherein the mixture comprising the milk-based substrate and the peptide exhibiting beta-galactosidase activity is heated to a temperature of at least 60° C. for at least four seconds before or after incubating the mixture at a temperature from 1° C.-10° C.

In a further embodiment the present invention relates to the use of a peptide or dimeric peptide exhibiting beta-galactosidase activity for producing a dairy product with reduced lactose content at a temperature from 1° C.-10° C. for a period of time sufficient to reduce the lactose concentration in the mixture to less than 0.2 g/L, wherein the peptide or dimeric peptide exhibiting beta-galactosidase activity is:
(i) a peptide having an amino acid sequence selected from SEQ ID NO: 22, 33, 14, 7, 9, 11, 30 and 1 or a peptide having an amino acid sequence identity of more than 85% to any of these sequences;
(ii) a peptide having an amino acid sequence selected from SEQ ID NO: 22, 33, 14, 13, 19, 7, 9, 11, 26 and 27, 30 and 1 or a peptide having an amino acid sequence identity of more than 85% to any of these sequences;
(iii) a peptide having an amino acid sequence represented by SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or enzymatically active fragments thereof, or an amino acid sequence of any one thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.

### LEGENDS TO THE FIGURES

FIG. 1. The specific activity of the purified enzymes determined at pH 6.7 at 37° C. with lactose as substrate, described as SUAL-1, discussed in example 6. The measured standard deviation at the given condition was less than 6%.

FIG. 2. The specific activity of the purified enzymes determined at pH 6.7 at 37° C. in presence of galactose, described as SUAG, discussed in example 7. The measured standard deviation at the given condition was less than 15%.

FIG. 3. The specific activity of the purified enzymes determined at pH 6.7 at 4° C. with lactose as substrate, described as SUAL-2, discussed in example 8. The measured standard deviation at the given condition was less than 5%.

FIG. 4. The specific activity of the purified enzymes determined at pH 6.7 at 43° C. with lactose as substrate, described as SUAL-3, discussed in example 9. The measured standard deviation at the given condition was less than 5%.

FIG. 5. The specific activity of the purified enzymes determined at pH 5.5 at 4° C. with lactose as substrate, described as SUAL-4, discussed in example 10. The measured standard deviation at the given condition was less than 5%.

FIG. 6. The specific activity of the purified enzymes determined at pH 5.5 at 37° C. with lactose as substrate, described as SUAL-5, discussed in example 11. The measured standard deviation at the given condition was less than 5%.

FIG. 7. The specific activity of the purified enzymes determined at pH 5.5 at 43° C. with lactose as substrate, described as SUAL-6, discussed in example 12. The measured standard deviation at the given condition was less than 5%.

FIG. 8. The specific activity of the purified enzymes determined at pH 4.5 at 4° C. with lactose as substrate, described as SUAL-7, discussed in example 13. The measured standard deviation at the given condition was less than 5%.

FIG. 9. The specific activity of the purified enzymes determined at pH 4.5 at 37° C. with lactose as substrate, described as SUAL-8, discussed in example 14. The measured standard deviation at the given condition was less than 5%.

FIG. 10. The specific activity of the purified enzymes determined at pH 4.5 at 43° C. with lactose as substrate, described as SUAL-9, discussed in example 15. The measured standard deviation at the given condition was less than 5%.

FIG. 11. The percentage residual lactose in the pasteurized milk, after the treatment with a fixed amount of the enzyme, after 24 hr at 5° C. determined using HPLC.

FIG. 12. The percentage residual lactose in the UHT milk, after the treatment with a fixed amount of the enzyme, after 24 hr at 25° C. determined using HPLC.

FIG. 13. The percentage residual activity of the purified enzymes at elevated temperatures, determined using lactose as substrate. The activity at pH 6.7 at 37° C. was considered as 100%.

FIG. 14. The percentage residual lactose present in pasteurized milk after incubation with lactase enzymes at different temperatures, at 37° C., 55° C. or 60° C. The detection limit of the LactoSens® kit used in the assay is either 0.01% to 0.2% or 0.02%-1.0% lactose.

FIG. 15. The percentage residual lactose present in pasteurized milk after incubation with lactase enzymes in a concentration of 0.047 mg/ml. The detection limit of the LactoSens® kit used in the assay is either 0.01% to 0.2% or 0.02%-1.0% lactose.

FIG. 16. The percentage residual lactose present in pasteurized milk incubated with lactase enzymes for a different reaction time, namely 15 or 30 minutes. The detection limit of the LactoSens® kit used in the assay is either 0.01% to 0.2% or 0.02%-1.0% lactose.

FIG. 17. The percentage residual lactose present in pasteurized milk incubated with lactase enzymes at different enzyme doses, namely 0.047 mg/ml or 0.024 mg/ml. The detection limit of the LactoSens® kit used in the assay is either 0.01% to 0.2% or 0.02%-1.0% lactose.

FIG. 18. The percentage residual lactose present in pasteurized milk incubated with lactase enzymes using a different dose and a different reaction time. The detection limit of the LactoSens® kit used in the assay is either 0.01% to 0.2% or 0.02%-1.0% lactose.

FIG. 19. The percentage residual lactose present in filtered milk incubated with lactase enzymes at 55° C. The detection limit of the LactoSens® kit used in the assay is either 0.01% to 0.2% or 0.02%-1.0% lactose.

FIG. 20. The percentage residual lactose present in filtered milk incubated with lactase enzymes at 55° C. and at different enzyme doses. The detection limit of the LactoSens® kit used in the assay is either 0.01% to 0.2% or 0.02%-1.0% lactose.

FIG. 21. The percentage residual lactose present in filtered milk incubated with lactase enzymes at 55° C. for a different reaction time. The detection limit of the LactoSens® kit used in the assay is either 0.01% to 0.2% or 0.02%-1.0% lactose.

FIG. 22. The kinetics of lactose hydrolysis in pasteurized milk at 4° C. with Ha-Lactase and NOLA® Fit with 50 mg/L dose. The enzyme was mixed in milk and stored at 4C for different time interval. The residual lactose was determined using LactoSens® assay kit (Chr. Hansen, Denmark). The detection limit of the LactoSens® kit used in the assay is either 0.01% to 0.2% or 0.02%-1.0% lactose.

FIG. 23. The percentage residual lactose measured after 12 hr and 24 hr of enzymes addition. The enzyme was mixed in milk and stored at 4° C. for different time interval. The residual lactose was determined using LactoSens® assay kit (Chr. Hansen, Denmark). The detection limit of the LactoSens® kit used in the assay is either 0.01% to 0.2% or 0.02%-1.0% lactose.

FIG. 24. The kinetics of lactose hydrolysis in pasteurized milk at 4° C. with novel lactases with 0.050 mg/mL dose. The enzyme was mixed in milk and stored at 4° C. for different time interval. The residual lactose was determined using LactoSens® assay kit (Chr. Hansen, Denmark). The NOLA® Fit and Ha-Lactase were used as controls. The detection limit of the LactoSens® kit used in the assay is either 0.01% to 0.2% or 0.02%-1.0% lactose.

FIG. 25. The kinetics of lactose hydrolysis in pasteurized milk at 4° C. with selected novel lactases with 0.050 mg/L dose. The measured residual lactose values are shown in the graph. The enzyme was mixed in milk and stored at 4° C. for different time interval. The residual lactose was determined using LactoSens® assay kit (Chr. Hansen, Denmark). The NOLA® Fit and Ha-Lactase were used as controls. The measured residual lactose values are shown in the graph. The detection limit of the LactoSens® kit used in the assay is either 0.01% to 0.2% or 0.02%-1.0% lactose.

FIG. 26. The effect of enzyme dose on lactose hydrolysis. The milk was incubated with different enzyme doses, mixed and stored at 4° C. for 24 hr. The residual lactose was determined using LactoSens® assay kit (Chr. Hansen, Denmark). The detection limit of the LactoSens® kit used in the assay is either 0.01% to 0.2% or 0.02%-1.0% lactose.

FIG. 27. Comparison of enzyme performance in different milk types. The milk was incubated with 0.052 mg/L in pasteurized and filtered milk, mixed and stored at 4° C. for 24 hr. The residual lactose was determined using LactoSens® assay kit (Chr. Hansen, Denmark). The detection limit of the LactoSens® kit used in the assay is either 0.01% to 0.2% lactose.

FIG. 28. The measured specific activity of purified enzymes determined at pH 6.7 at different temperatures. The specific activity values were defined as μmole of glucose formed per minute per milligram of enzyme under a given condition. The measured standard deviations at the given conditions were between 5-20%.

FIG. 29. The measured specific activity of purified enzymes determined at pH 5.5 at different temperatures. The specific activity values were defined as μmole of glucose formed per minute per milligram of enzyme under a given condition. The measured standard deviations at the given conditions were around 5%.

FIG. 30. The measured specific activity of purified enzymes determined at pH 4.5 at different temperatures. The specific activity values were defined as μmole of glucose formed per minute per milligram of enzyme under a given condition. The measured standard deviations at the given conditions were around 5%.

### DETAILED DISCLOSURE OF THE INVENTION

The present inventors have found that certain peptides and dimeric peptides exhibiting beta-galactosidase enzyme activity are surprisingly stabile at many different physical conditions giving a relatively high activity outside of the ranges normally seen to be optimal for this class of enzymes.

Accordingly, these by the present inventors identified enzymes have a relatively high activity around 4° C. or 5° C. and may thus be used for lactose hydrolysis in the production of e.g. fresh milk. The novel enzymes are thus particularly suitable for reducing the lactose content of milk-based products, such as dairy products, at low temperatures.

A further advantage of these novel improved peptides exhibiting beta-galactosidase enzyme activity is that they have a relatively low degree of galactose inhibition. The lower galactose inhibition of these novel enzymes is highly relevant for applications wherein very low lactose concentrations are desired.

In terms of applicability for fermented products it is highly advantageous that the enzymes as described herein have a high beta-galactosidase enzymatic activity at a relatively broad temperature range of between 4° C. and 43° C., such as around 37° C., where fermentation would normally be optimal, but also that this activity of the beta-galactosidase enzyme is present at low pH, such as down to 4.5, or down to 4.0, or down to 3.5, or even down to pH 3.

In summary, it has been found by the present inventors that some peptides exhibiting beta-galactosidase enzyme activity is active over wide range of temperature, active over wide range of pH, has a general high hydrolytic activity without side activities, that these peptides have no or little galactose inhibition, such as less than 60%, and that they are stable over long-term storage.

The beta-galactosidase activity may be determined by measuring the amount of released glucose after incubation with lactose at set conditions. Released glucose can be detected by a coloring reaction.

### Definitions

The term “milk”, as used herein and in the context of the present invention, is to be understood as the lacteal secretion obtained by milking any mammal, such as cow, sheep, goats, buffalo or camel.

The term “composition containing lactose” as used herein refers to any composition, such as any liquid that contain lactose in significant measurable degree, such as a lactose content higher than 0.002% (0.002 g/100 ml). Encompassed within this term are milk and milk-based substrates.

The term “milk-based substrate”, in the context of the present invention, may be any raw and/or processed milk material. Useful milk-based substrates include, but are not limited to solutions/suspensions of any milk or milk like products comprising lactose, such as whole or low fat milk, skim milk, buttermilk, low-lactose milk, reconstituted milk powder, condensed milk, solutions of dried milk, UHT milk, whey, whey permeate, acid whey, cream, fermented milk products, such as yoghurt, cheese, dietary supplement and probiotic dietary products. Typically the term milk-based substrate refers to a raw or processed milk material that is processed further in order to produce a dairy product.

The term “pasteurization” as used herein refers to the process of reducing or eliminating the presence of live organisms, such as microorganisms in a milk-based substrate. Preferably, pasteurization is attained by maintaining a specified temperature for a specified period of time. The specified temperature is usually attained by heating. The temperature and duration may be selected in order to kill or inactivate certain bacteria, such as harmful bacteria, and/or to inactivate enzymes in the milk. A rapid cooling step may follow.

The term “dairy product” as used herein may be any food product wherein one of the major constituents is milk-based. Usually the major constituent is milk-based and in some embodiments, the major constituent is a milk-based substrate which has been treated with an enzyme having beta-galactosidase activity according to a method of the present invention.

A dairy product according to the invention may be, e.g., skim milk, low fat milk, whole milk, cream, UHT milk, milk having an extended shelf life, a fermented milk product, cheese, yoghurt, butter, dairy spread, butter milk, acidified milk drink, sour cream, whey based drink, ice cream, condensed milk, dulce de leche or a flavored milk drink.

A dairy product may additionally comprise non-milk components, e.g. vegetable components such as, e.g., vegetable oil, vegetable protein, and/or vegetable carbohydrates. Dairy products may also comprise further additives such as, e.g., enzymes, flavoring agents, microbial cultures such as probiotic cultures, salts, sweeteners, sugars, acids, fruit, fruit prep, fruit juices, or any other component known in the art as a component of, or additive to, a dairy product.

The terms “fermented dairy product” or “fermented milk product” as used herein is to be understood as any dairy product wherein any type of fermentation forms part of the production process. Examples of fermented dairy products are products like yoghurt, buttermilk, creme fraiche, quark and fromage frais. A fermented dairy product may be produced by or include steps of any method known in the art.

The term “fermentation” as used herein refers to the conversion of carbohydrates into alcohols or acids through the action of a microorganism. In some embodiments fermentation according to the present invention comprises the conversion of lactose to lactic acid. In the context of the present invention, “microorganism” may include any bacterium or fungus being able to ferment the milk substrate.

The term “increased beta-galactosidase enzyme activity” as used herein refers to a relatively higher specific activity of a beta-galactosidase enzyme in comparison to a reference sequence.

The term “peptide exhibiting beta-galactosidase enzyme activity” as used herein refers to any peptide, which has enzymatic activity to catalyze the hydrolysis of the disaccharide lactose into its component monosaccharides glucose and galactose. This peptide may also be referred to as a lactase or simply a beta-galactosidase (EC: 3.2.1.23).

In a preferred embodiment the beta-galactosidase activity is determined by incubating 13 μl of a solution comprising a known amount of a purified lactase enzyme with a solution comprising 140 mM of lactose at pH 6.7 and 37° C. for 10 min, terminating the lactase reaction by increasing the temperature to 95° C. for 10 min. The amount of glucose formed was determined by incubating the reaction product at 30° C. for 40 min with a 80 μL solution of glucose oxidase (0.6 g/L), 2,2′-azino-bis(3-ethylbenzothiazoline-6-sulfonic acid diammonium salt) (1.0 g/LABTS) and horseradish peroxidase (0.02 g/L) and determining the absorbance at 610 nm using a FLUOphotometer. The absorbance is correlated to the concentration of glucose formed per minute and the maximum value determined (in μmol of glucose formed/min) is determined as the Unit of Lactase Activity 1 (also designated herein UAL-1). The Specific Activity of Lactase (also herein designated SUAL-1) at pH 6.7 at 37° C. is defined as μmol of glucose formed/min/mg of enzyme and is determined by dividing UAL-1 by the lactase protein concentration in mg. Full details of a preferred alternative of carrying out this assay are illustrated in Example 6.

While characterizing beta-galactosidase activity by reference to values of the unit μmol of glucose formed/min/mg of enzyme represents the standard approach for the determination of the activity, other units may equally be used to characterize the activity of the lactase enzymes using the above test. Accordingly, some of the examples characterize the lactase enzyme activity by reference to μM of glucose formed per second per μM of enzyme.

In alternative embodiments the assay can be carried out using a different temperature or different pH values for the lactase incubation.

The terms “peptide” and “oligopeptide” as used in the context of this present application are considered synonymous (as is commonly recognized) and each term can be used interchangeably as the context requires to indicate a chain of at least two amino acids coupled by peptidyl linkages. The word “polypeptide” is used herein for chains containing more than ten amino acid residues. All peptide and polypeptide formulas or sequences herein are written from left to right and in the direction from amino terminus to carboxy terminus. “Proteins” as used herein refers to peptide sequences as they are produced by some host organism and may include posttranslational modification, such as added glycans.

The terms “amino acid” or “amino acid sequence,” as used herein, refer to an oligopeptide, peptide, polypeptide, or protein sequence, or a fragment of any of these, and to naturally occurring or synthetic molecules. In this context, “fragment” refer to fragments of a peptide exhibiting beta-galactosidase enzyme activity, which retain some enzymatic activity. Where “amino acid sequence” is recited herein to refer to an amino acid sequence of a naturally occurring protein molecule, “amino acid sequence” and like terms are not meant to limit the amino acid sequence to the complete native amino acid sequence associated with the recited peptide molecule.

Exemplary peptides of the invention also include fragments of at least about 50,100,150, 200, 250, 300, 350, 400, 450, 500, 550, 600, 650, 700, 750, 800 or more residues in length, or over the full length of an enzyme. Accordingly a “peptide fragment” or “enzymatically active fragment” of the invention are fragments that retain at least some functional enzymatic activity. Typically a peptide fragment of the invention will still contain the functional catalytic domain or other essential active sites of the peptide exhibiting beta-galactosidase enzyme activity. Other domains may be deleted.

Typically, the specific beta-galactosidase enzyme activity will be measured and indicated as μmol of glucose formed/min/mg of enzyme used. This specific value however will vary depending on conditions applied, such as temperature, and pH. Accordingly, values for beta-galactosidase enzyme activity may also be referred to as relative to a reference known enzyme, such as the beta-galactosidase enzyme defined by SEQ ID NO:34 OR SEQ ID NO:35.

Unless otherwise stated the term “Sequence identity” for amino acids as used herein refers to the sequence identity calculated as (n_{ref}-n_{dif})·100/n_{ref}, wherein n_{dif} is the total number of non-identical residues in the two sequences when aligned and wherein n_{ref} is the number of residues in one of the sequences.

In some embodiments the sequence identity is determined by conventional methods, e.g., Smith and Waterman, 1981, Adv. Appl. Math. 2:482, by the search for similarity method of Pearson & Lipman, 1988, Proc. Natl. Acad. Sci. USA 85:2444, using the CLUSTAL W algorithm of Thompson et al., 1994, Nucleic Acids Res 22:467380, by computerized implementations of these algorithms (GAP, BESTFIT, FASTA, and TFASTA in the Wisconsin Genetics Software Package, Genetics Computer Group). The BLAST algorithm (Altschul et al., 1990, Mol. Biol. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

A peptide with a specific amino acid sequence as described herein may vary from a reference peptide sequence by any of amino acid substitutions, additions/insertions, or deletions.

Some embodiments according to the present invention refer to the use of a peptide with an amino acid sequence represented by SEQ ID NO: 1-33 or a sequence with at least 80% sequence identity to any one of said sequences. In some embodiments this sequence identity may be at least about 81%, 82%, 83%, 84%, 85%, 86%, 87%, 88%, 89%, 90%, 91%, 92%, 93%, 94%, 95%, 96%, 97%, 98%, or 99%, such as a peptide with not more than 1, 2, 3, 4, 5, 6, 7, 8, 9, 10, 11, 12, 13, 14, 15, 16, 17, 18, 19, 20, 21, or 22 amino acid substitutions, additions or deletions as compared to any one reference amino acid sequence represented by SEQ ID NO: 1-33. The invention also features biologically active fragments of the peptides according to the invention. Biologically active fragments of a peptide of the invention include peptides comprising amino acid sequences sufficiently identical to or derived from the amino acid sequence of peptide of the invention which include fewer amino acids than the full length protein but which exhibit a substantial part of the biological activity of the corresponding full-length peptide. Typically, biologically active fragments comprise a domain or motif with at least one activity of a variant protein of the invention. A biologically active fragment of a peptide of the invention can be a peptide which is, for example, 100, 200, 300, 400, 500, 600, 700, 800, 900, 1000 or more amino acids in length.

The term “host cell”, as used herein, includes any cell type which is susceptible to transformation, transfection, transduction, and the like with a nucleic acid construct or expression vector comprising a polynucleotide encoding the peptides of the present invention. A host cell may be the cell type, where a specific enzyme is derived from or it may be an alternative cell type susceptible to the production of a specific enzyme. The term includes both wild type and attenuated strains.

Suitable host cell may be any bacteria including lactic acid within the order “Lactobacillies” which includes Lactococcus spp., Streptococcus spp., Lactobacillus spp., Leuconostoc spp., Pseudoleuconostoc spp., Pediococcus spp., Brevibacterium spp., Enterococcus spp. and Propionibacterium spp. Also included are lactic acid producing bacteria belonging to the group of anaerobic bacteria, bifidobacteria, i.e. Bifidobacterium spp., which are frequently used as food cultures alone or in combination with lactic acid bacteria. Also included within this definition are Lactococcus lactis, Lactococcus lactis subsp. cremoris, Leuconostoc mesenteroides subsp. cremoris, Pseudoleuconostoc mesenteroides subsp. cremoris, Pediococcus pentosaceus, Lactococcus lactis subsp. lactis biovar. diacetylactis, Lactobacillus casei subsp. casei and Lactobacillus paracasei subsp. Paracasei and thermophilic lactic acid bacterial species include as examples Streptococcus thermophilus, Enterococcus faecium, Lactobacillus delbrueckii subsp. lactis, Lactobacillus helveticus, Lactobacillus delbrueckii subsp. bulgaricus and Lactobacillus acidophilus. Other specific bacteria within this definition includes bacteria of the family Bifidobacteriaceae, such as from the genus Bifidobacterium, such as from a strain of bifidobacterium animalis or bifidobacterium longum, bifidobacterium adolescentis, bifidobacterium bifodum, bifidobacterium breve, bifidobacterium catenulatum, bifidobacterium infantus or from the genus Lactobacillus, such as L. sakei, L. amylovorus, L. delbrueckii subsp. Lactis, and L. helveticus.

Also included within this definition of host cells include strain of Agaricus, e.g. A. bisporus; Ascovaginospora; Aspergillus, e.g. A. niger, A. awamori, A. foetidus, A. japonicus, A. oryzae; Candida; Chaetomium; Chaetotomastia; Dictyostelium, e.g. D. discoideum; Kluveromyces, e.g. K. fragilis, K. lactis; Mucor, e.g. M. javanicus, M. mucedo, M. subtilissimus; Neurospora, e.g. N. crassa; Rhizomucor, e.g. R. pusillus; Rhizopus, e.g. R. arrhizus, R. japonicus, R. stolonifer; Sclerotinia, e.g. S. libertiana; Torula; Torulopsis; Trichophyton, e.g. T. rubrum; Whetzelinia, e.g. W. sclerotiorum; Bacillus, e.g. B. coagulans, B. circulans, B. megaterium, B. novalis, B. subtilis, B. pumilus, B. stearothermophilus, B. thuringiensis; Bifidobacterium, e.g. B. Iongum, B. bifidum, B. animalis; Chryseobacterium; Citrobacter, e.g. C. freundii; Clostridium, e.g. C. perfringens; Diplodia, e.g. D. gossypina; Enterobacter, e.g. E. aerogenes, E. cloacae Edwardsiella, E. tarda; Erwinia, e.g. E. herbicola; Escherichia, e.g. E. coli; Klebsiella, e.g. K. pneumoniae; Miriococcum; Myrothesium; Mucor; Neurospora, e.g. N. crassa; Proteus, e.g. P. vulgaris; Providencia, e.g. P. stuartii; Pycnoporus, e.g. Pycnoporus cinnabarinus, Pycnoporus sanguineus; Ruminococcus, e.g. R. torques; Salmonella, e.g. S. typhimurium; Serratia, e.g. S. liquefasciens, S. marcescens; Shigella, e.g. S. flexneri; Streptomyces, e.g. S. antibioticus, S. castaneoglobisporus, S. violeceoruber; Trametes; Trichoderma, e.g. T. reesei, T. viride; Yersinia, e.g. Y. enterocolitica.

To produce lactose free milk pasteurized milk (<0.01% residual lactose level) at cold temperatures (4-5° C.) in 24 hr, the recommended dose of the Ha-Lactase and NOLA® are 55-70 mg/L (10000 NLU/L) and 45-60 mg/L respectively (10000 BLU/L), respectively. The enzymes of the present invention provided very low residual lactose concentrations at low temperatures (<0.01% to 0.2%). The specific activity measurements shows that the novel enzymes have 2-5 higher activity than Ha-Lactase and NOLA® Fit, therefore they will require lesser time to produce the lactose free milk.

The Examples below show that the novel lactases are faster than Ha-Lactase and NOLA® Fit and results in lactose free pasteurized milk in significantly shorter time. These new enzymes can reduce the overall process time. Additionally, with novel enzymes it is possible to further reduce the enzyme dose between 25-50% to produce lactose free/reduced pasteurized milk.

TABLE 1
The gene numbers with corresponding sequence identification number.

| Gene number | Sequence Identity number | Species name |
|---|---|---|
| G4 | SEQ ID No 1 | Bifidobacterium adolescentis |
| G16 | SEQ ID No 2 (domain a) | Lactobacillus sakei |
| | SEQ ID No 3 (domain b) | |
| G35 | SEQ ID No 4 | Bifidobacterium adolescentis |
| G40 | SEQ ID No 5 (domain a) | Lactobacillus amylovorus |
| | SEQ ID No 6 (domain b) | |
| G44 | SEQ ID No 7 | Bifidobacterium bifidum |
| G51 | SEQ ID No 8 | Bifidobacterium bifidum |
| G57 | SEQ ID No 9 | Bifidobacterium breve |
| G62 | SEQ ID No 10 | Bifidobacterium catenulatum |
| G66 | SEQ ID No 11 | Bifidobacterium catenulatum |
| G83 | SEQ ID No 12 | Lactobacillus delbrueckii subsp. |
| | | bulgaricus |
| G84 | SEQ ID No 13 | Lactobacillus delbrueckii subsp. |
| | | lactis |
| G95 | SEQ ID No 14 | Lactobacillus delbrueckii subsp. |
| | | bulgaricus |
| G100 | SEQ ID No 15 | Lactobacillus delbrueckii subsp. |
| | | bulgaricus |
| G104 | SEQ ID No 16 | Lactobacillus delbrueckii subsp. |
| | | lactis |
| G108 | SEQ ID No 17 | Lactobacillus delbrueckii subsp. |
| | | bulgaricus |
| G109 | SEQ ID No 18 | Lactobacillus delbrueckii subsp. |
| | | bulgaricus |
| G118 | SEQ ID No 19 | Lactobacillus delbrueckii subsp. |
| | | lactis |
| G145 | SEQ ID No 20 (domain a) | Lactobacillus helvaticus |
| | SEQ ID No 21 (domain b) | |
| G158 | SEQ ID No 22 | Bifidobacterium longum |
| G224 | SEQ ID No 23 (domain a) | Lactobacillus reuteri |
| | SEQ ID No 24 (domain b) | |
| G256 | SEQ ID No 25 | Lactobacillus delbrueckii subsp. |
| | | lactis |
| G282 | SEQ ID No 26 (domain a) | Lactobacillus helvaticus |
| | SEQ ID No 27 (domain b) | |
| G334 | SEQ ID No 28 (domain a) | Lactobacillus crispatus |
| | SEQ ID No 29 (domain b) | |
| G335 | SEQ ID No 30 | Streptococcus thermophilus |
| G336 | SEQ ID No 31 | Lactobacillus delbrueckii subsp. |
| | | indicus |
| G11 | SEQ ID No 32 | Bifidobacterium adolescentis |
| G33 | SEQ ID No 33 | Bifidobacterium adolescentis |
| G600 | SEQ ID No 34 | Bifidobacterium bifidum |
| G500 | SEQ ID No 35 | Kluyveromyces lactis |

### EXAMPLES

### General Material and Methods

### Molecular Cloning and Genetic Techniques

Techniques for restriction enzyme digestions, ligation, transformation and other standard molecular biology manipulations were based on methods described in the literature (Maniatis et al. “Molecular cloning: a laboratory manual, 2nd edition” Cold Spring Harbor Laboratory Press, Cold Spring Harbor, NY, 1989; Sambrook and Russell “Molecular Cloning: A Laboratory Manual, 3rd edition” Cold Spring Harbor Laboratory Press, Cold Spring Harbor, NY 2001; Miller “Experiment in molecular genetics” Cold Spring Harbor Laboratory Press, 1972); or as suggested by the manufacturer. The PCR was carried out in a DNA thermal cycler obtained from (Bio-Rad, USA). DNA sequencing was performed by LGC, Berlin, Germany. Proteins were analyzed by polyacrylamide gel electrophoresis (PAGE) under the denaturation conditions using sodium dodecyl sulphate on gels containing 10% SDS (Mini-PROTEAN® TGX stain-free™ gel, Biorad, USA). Protein concentrations were determined using BCA method by following the protocol supplied with the kit.

### Bacterial Strains, Plasmid and Growth Conditions

Escherichia coli strain TOP10 (Invitrogen) was used for the cloning and isolation of plasmids. The beta-galactosidase deficient E. coli strain BW25113 (Δ(araD-araB)567, ΔlacZ4787(::rrnB-3), λ-, rph-1, Δ(rhaD-rhaB)568, hsdR514) (Datsenko K A, Wanner B L; 2000, Proc Natl Acad Sci U.S.A. 97:6640-6645) was used in combination with the pBAD/His vector (obtained from Invitrogen™ Life Technologies Corporation Europe BV) for recombinant protein production.

### Growth Media for Protein Expression

2×PY medium containing (16 g/L BD BBL™ Phyton™ Peptone, 10 g/L Yeast Extract, 5 g/L NaCl) was used for the recombinant protein production. The growth medium was supplemented with ampicillin (100 μg/ml) to maintain the plasmid. Protein production was initiated by adding 0.05% of arabinose in to the culture medium.

### Example 1

### Construction of the Expression Vector for the Production of Lactases

The genomic DNA of the lactic acid bacteria or bifidobacteria was extracted using commercial genomic extraction kit by following the supplied protocol (DNeasy, Qaigen, Germany). The lactase gene was amplified by PCR using two synthetic primers, using the purified genomic DNA source as biomass, and the PCR reagents were supplied in the Phusion U Hot start DNA polymerase (Thermo Scientific, USA) kit. The lactase gene was cloned into the start codon of the expression vector pBAD/His using the USER cloning method (Nour-Eldin H H, Geu-Flores F, Halkier B A, Plant Secondary Metabolism Engineering, Methods in Molecular Biology, 643; 2010), resulting in the expression construct. With the USER cloning method long, complementary overhangs in both PCR product and destination vector were generated. These overhangs can anneal to each other to form a stable hybridization product which was used to transform into E. coli without ligation. For the generation of overhangs in the PCR product, a single deoxyuradine residue is included in the upstream region of each primer to amplify target DNA. The lactase gene was amplified using the forward primer (5′-ATTAACCAUGCGACGCAACTTCGAATGGCC-3′) (SEQ ID NO: 36) and reverse primer (ATCTTCTCUTTACCGCCTTACCACGAGCACG) (SEQ ID NO: 37) containing a uridine at 9th position (as shown in bold), followed by the lactase gene sequence. In parallel, the vector DNA was PCR amplified using the forward (5′-AGAGAAGAUTTTCAGCCTGATACAGATTAAATC-3′) (SEQ ID NO: 38) and reverse primer (5′-ATGGTTAAUTCCTCCTGTTAGCCCAAAAAACGG-3′) (SEQ ID NO: 39) pair containing single deoxyuracil residue at 9th positions (as highlighted in bold) followed by vector DNA sequence. The PCR products were purified using the commercial PCR purification kit (Qiagen, Denmark). The purified PCR products (lactase gene and the vector DNA) were mixed in equimolar amount and incubated with a commercial USER enzyme mix (New England Biolabs, USA) by following the supplied protocol. These enzymes remove the uracil residue and also the short fragment upstream of the uridine, thereby creating complementary overhang in the PCR products. These complementary overhangs anneal with each other resulting in the pBAD-lactase expression vector. Aliquots of the ligation mixture were transformed into chemically competent E. coli TOP 10 cells. Transformants were selected at 37° C. on L B-Amp plates (L B; Luria-Bertani, Amp; 100 μg/ml ampicillin). The following day, colony PCR was carried out using a small biomass from the overnight grown transformant using the vector primers (primer 1; 5′-CGGCGTCACACTTTGCTATGCC-3′ (SEQ ID NO: 40) and primer 2; 5′-CCGCGCTACTGCCGCCAGGC-3′ (SEQ ID NO: 41)). The positive clones from the colony PCR were cultured in 5 mL LB-Amp medium and plasmid DNA was isolated from the cells. The cloned lactase gene was sequenced to verify that no additional mutations had been introduced during the amplification of the gene. The plasmid DNA was transformed in to the expression host E. coli strain BW25113.

### Example 2

### Expression of Lactases in E. coli Expression Host

The lactase enzyme was produced in E. coli BW25113 using the pBAD expression system. Freshly transformed E. coli BW25113 cells carrying the plasmid DNA were collected from a Lb-Amp plate using a sterile loop and used to inoculate 5 mL of Lb-Amp medium. The overnight grown culture (200 μL) was used to inoculate 50 mL 2×PY medium (containing 100 μg/mL ampicillin) in a 250 mL flask in a shaker (Innova® 42). The culture was grown at 37° C. at 220 rpm until the OD600 reached between 0.6-0.8. The lactase expression was initiated by adding 0.05% arabinose into the culture medium and the cells were cultured for additional 16-20 hours at 18° C. at 180 rpm. Cells were harvested by centrifugation (5000 rpm, 10 min at 4°° C.) and were stored at −20° C. until further use.

### Example 3

### Protein Purification Using Immobilized Metal Affinity Chromatography

Cells from 50 mL culture was thawed on ice and the cells were lysed using 10 mL mixture of lysis buffer (BugBuster® (Novagen) containing 2 mg/mL Lysozyme (Sigma Aldrich), 1 unit Benzonase (Sigma Aldrich), and 1× Complete Protease inhibitor cocktail (EDTA-free, Roche)) by incubating the cells at room temperature for 30 min. After 30 min, the cell debris was removed by centrifugation at 16000 rpm for 20 min at 4° C. The obtained supernatant was filtered through 0.45μm pore diameter filter. A gravity flow Ni-Sepharose (GE Healthcare) column was prepared with 1 mL slurry by washing out the ethanol and water. The column was then equilibrated with washing buffer (50 mM of NaH₂PO₄, pH 8.0 containing 300 mM of NaCl and 20 mM of Imidazole). The cell-free extract was applied to the column and the non-bound proteins were eluted from the column. The column was washed with 20 mL of washing buffer and the retained proteins were eluted with 3.5 mL of elution buffer (50 mM of NaH₂PO₄, pH 8.0 containing 300 mM of NaCl and 250 mM of imidazole). The collected fractions were analyzed by SDS-PAGE on gels containing 10% acrylamide and those contained the purified lactase enzymes combined together. The buffer was exchanged against the storage buffer (50 mM KH₂PO₄ buffer pH 7.0 containing 10 mM NaCl, 1 mM MgCl₂), using a prepacked PD-10 desalting G-25 gel filtration column (GE Healthcare). The purified enzymes were stored at 4° C. until further use.

### Example 4

### Protein Purification Using Gel Filtration Chromatography

Cells from 50 mL culture was thawed on ice and the cells were lysed using 10 mL mixture of lysis buffer (BugBuster® (Novagen) containing 2 mg/ml lysozyme, 1 unit Benzonase (Sigma Aldrich), and 1× Complete Protease inhibitor cocktail (EDTA-free, Roche)) by incubating the cells at room temperature (25° C.) for 30 min. After 30 min, the cell debris was removed by centrifugation at 16000 rpm for 20 min at 4° C. The obtained supernatant was filtered through 0.45 μm pore diameter filter. The clear cell-free extract was concentrated by filtering through a 30000 Dalton filter (Vivaspin 20, GE Healthcare) by following the supplied protocol. A gravity flow Sephadex G50 superfine (Pharmacia Chemicals, Sweden) column was prepared with 1 g of column material (prepared by boiling in 100 ml water for 1 hour, cooled to room temperature). A column was prepared by applying 20 ml of the cooled slurry on a 30 mL filtration column. The column was washed with MilliQ water and equilibrated with wash buffer B (50 mM of NaH₂PO₄ buffer, pH 7.0). 500 μL of the concentrated supernatant was applied on the column and allowed the supernatant to enter in the column bed. The wash buffer (50 mM of NaH₂PO₄ buffer, pH 7.0) was applied on top of the column and the eluent fractions were collected individually. The collected fractions were analyzed on SDS-PAGE gel (containing 10% acrylamide). The protein fractions were combined together and buffer was exchanged against the storage buffer (50 mM KH₂PO₄ buffer pH 7.0 containing 10 mM NaCl, 1 mM MgCl₂) with the desalting column as described in earlier section. The purified enzymes were stored at 4° C. until further use.

### Example 5

### Protein Concentration Measurement Using BCA Assay

The concentration of purified lactases was determined using Pierce™ BCA protein assay kit (Thermo Fisher Scientific, Germany) by following the protocol supplied with the kit.

### Example 6

### Activity Determination Using Purified Enzymes on Lactose as Substrate at pH 6.7 at 37° C.

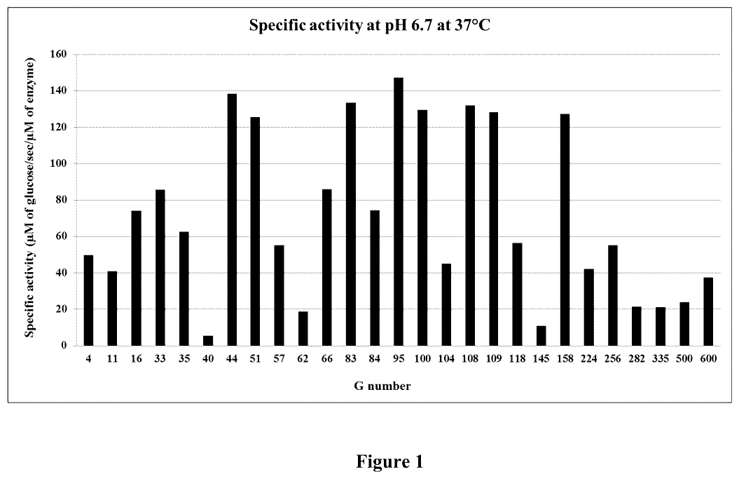

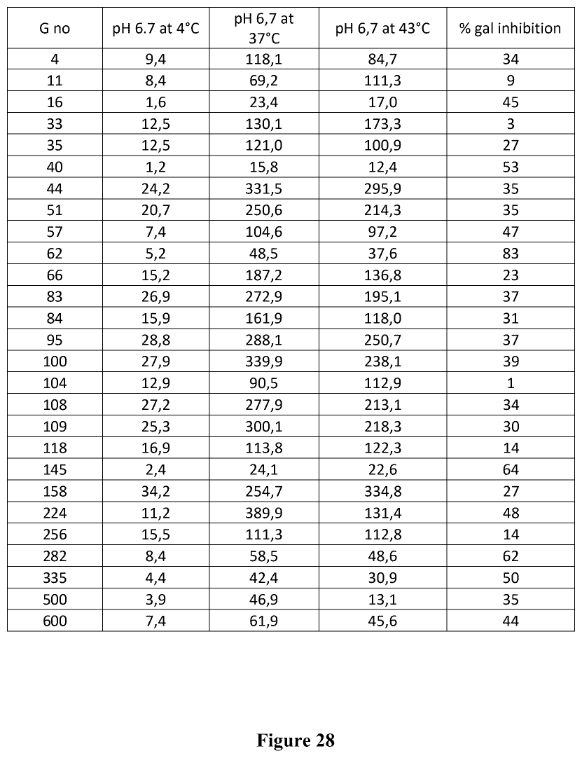

To measure the beta-galactosidase activity, the purified lactases were diluted to 40× in buffer A (50 mM NaH₂PO₄ buffer pH 6.7 containing 100 μM of MgSO₄). In a separate reaction, the diluted enzyme was incubated with lactose solution prepared in buffer B (140 mM of lactose prepared in 100 mM sodium-citrate buffer of pH 6.7, containing 100 μM of MgSO₄). The reaction mixture was prepared by mixing 13 μL of diluted enzyme and 37 μL of lactose solution in a PCR tube. The reaction mixture was incubated in a DNA thermal cycler with the following incubation parameters (reaction time; 10 min at 37° C., enzyme inactivation; 10 min at 95° C., cooling; 4° C.). The reaction mixtures were stored at-20° C. until further use. To determine the amount of glucose formed during the reaction, 10 μL of the reaction mixture was transferred to one well of standard microtiter plate (Thermo Fischer Scientific, Denmark) containing 80 82 L of buffer C (100 mM of NaH₂PO₄ buffer, pH 7.0, containing glucose oxidase; 0.6 g/L (Sigma Aldrich), 2,2′-azino-bis(3-ethylbenzothiazoline-6-sulfonic acid diammonium salt); ABTS: 1.0 g/L (Sigma Aldrich), horseradish peroxidase; 0.02 g/L (Sigma Adrich)) and incubated at 30° C. for 40 min. After 40 min, the absorbance was determined at 610 nm using FLUOStar Omega UV-plate reader (BMG Labtech, Germany). The absorbance values between 0.1 and 1.5 were used for calculations, if the A610 nm value>1.5, the reaction mixture was diluted up to 10× with buffer A. With each purified enzyme, the reactions were carried out in triplicate and the mean value of the triplicate measurement was used for calculation. The protein purification performed with the E. coli cells transformed with the empty pBAD/His was used for normalization. Using a known concentration of glucose (0-2.5 mM), a standard curve was drawn and the slope of the curve was used to calculate the glucose formed during the reaction. The maximum absorbance value for each lactase was used to determine μmol of glucose formed per min (for example by correlating the absorbance value to the glucose concentration formed using a standard or calibration curve) and is also designated Unit of Lactase Activity 1 (or UAL-1) at pH 6.7 at 37° C. The Specific Activity (designated as SUAL-1) at pH 6.7 at 37° C. is defined as μmol of glucose formed per min per mg of enzyme (μmol of glucose/min/mg of enzyme) and is determined by dividing UAL-1 by the protein concentration in mg. The specific activity of SEQ ID NO: 34 and SEQ ID NO: 35 were determined under essentially the same conditions. The high specific activity at pH 6.7 is highly desired for robustness for the enzyme in fresh and fermented milk applications. The detailed results of the specific activity of enzymes at pH 6.7 at 37° C. are described in FIG. 28. Additionally the activity was described as μM of glucose formed per second per μM of enzyme added. The results are shown in FIG. 1.

The specific activity of the enzymes was determined at pH 6.7 and at 37° C. and used to calculate the approximate time required for hydrolysis of lactose using a fixed enzyme dose based activity units at pH 6.7 at 37° C. and 140 mM lactose as substrate (SUAL-1). The results in terms of time 10 calculated for lactose hydrolysis are shown in Table 2:

TABLE 2

| | | Time required for complete | | |
|---|---|---|---|---|
| | | lactose hydrolysis using | | |
| | | 1 mg | 100 mg | 47 mg |
| | | enzyme | enzyme | enzyme |
| | | per liter | per | per |
| G No. | SUAL-1 | (m in) | liter (sec) | liter (sec) |
| 4 | 118.1 | 1185 | 711 | 1508 |
| 11 | 69.2 | 2023 | 1214 | 2573 |
| 16 | 23.4 | 5996 | 3597 | 7626 |
| 33 | 130.1 | 1076 | 646 | 1369 |
| 40 | 15.8 | 8874 | 5324 | 11287 |
| 44 | 331.5 | 422 | 253 | 537 |
| 57 | 104.6 | 1339 | 803 | 1703 |
| 66 | 187.2 | 748 | 449 | 951 |
| 83 | 272.9 | 513 | 308 | 653 |
| 84 | 161.9 | 865 | 519 | 1100 |
| 95 | 288.1 | 486 | 292 | 618 |
| 104 | 90.5 | 1548 | 929 | 1969 |
| 108 | 277.9 | 504 | 302 | 641 |
| 118 | 113.8 | 1230 | 738 | 1565 |
| 158 | 254.7 | 550 | 330 | 699 |
| 282 | 58.5 | 2392 | 1435 | 3042 |
| 335 | 42.4 | 3298 | 1979 | 4195 |
| 500 | 46.9 | 2983 | 1790 | 3794 |
| 600 | 61.9 | 2263 | 1358 | 2879 |

TABLE 2. Specific activity of purified enzymes determined at pH 6.7 at 37° C. with lactose as substrate, described SUAL-1, discussed in example 6. The calculated time required in seconds for the complete lactose hydrolysis. The measured standard deviation at the given condition was less than 6%. The theoretical time required to hydrolyze the 140 mmol of lactose is calculated by assuming that reaction rate stay unchanged over the entire reaction period

Note* Complete lactose hydrolysis is defined as the time required for the enzyme to hydrolyze 140 mmol of lactose using a fixed enzyme concentration based on specific activity units at pH 6.7 at 37° C. with 140 mmol lactose as substrate (SUAL).

### Example 7

### Activity Determination Using Purified Enzymes in the Presence of Galactose at pH 6.7 at 37° C.

The purified lactases were diluted to 40× in buffer A (50 mM NaH₂PO₄ buffer pH 6.7 containing 100 μM of MgSO₄). In separate reactions, the diluted enzymes were incubated with buffer D (140 mM of lactose and 140 mM of galactose prepared in 100 mM sodium-citrate buffer of pH 6.7, containing 100 μM of MgSO₄). The reaction mixture consists of 13 μL of the diluted enzyme and 37 μl of buffer D in a PCR tube. The reaction mixture was incubated in thermal cycler with the following incubation parameters (reaction time: 10 min at 37° C., enzyme inactivation: 10 min at 95° C., cooling: 4° C.). The reaction mixtures were stored at-20° C. until further use. To determine the amount of glucose formed during the reaction, 10 μL of the reaction mixture was transferred to one well of standard microtiter plate (Thermo Fischer Scientific, Denmark) containing 80 μl of buffer C (100 mM of NaH₂PO₄ buffer, pH 7.0, containing glucose oxidase; 0.6 g/L (Sigma Aldrich), 2,2′-azino-bis(3-ethylbenzothiazoline-6-sulfonic acid diammonium salt); ABTS: 1.0 g/L (Sigma Aldrich), horseradish peroxidase; 0.02 g/L (Sigma Adrich)) and incubated at 30° C. for 40 min. After 40 min, the absorbance was determined at 610 nm using FLUOStar Omega UV-plate reader (BMG Labtech, Germany). The absorbance values between 0.1 and 1.5 were used for calculations, if the A610 nm value>1.5, the reaction mixture was diluted up to 10× with buffer A. With each purified enzyme, the reactions were carried out in triplicate and the mean value of the triplicate measurement was used for calculation. The protein purification performed with the E. coli cells transformed with the empty pBAD/His was used for normalization. Using a known concentration of glucose (0-2.5 mM), a standard curve was drawn and the slope of the curve was used to calculate the absorbance corresponding to 1 μM of glucose formed during the reaction. The maximum absorbance value for each lactase was used to determine μM of glucose formed per sec, described as 1 Unit of Activity with Galactose at pH 6.7 at 37° C. (UAG). The specific activity at pH 6.7 at 37° C. in presence of galactose is defined as μM of glucose formed per second per μM of enzyme (μM of glucose/sec/μM of enzyme) and determined by dividing UAG by the protein concentration in μM, described as SUAG.

The percentage inhibition of enzymes with galactose is calculated by using the formula

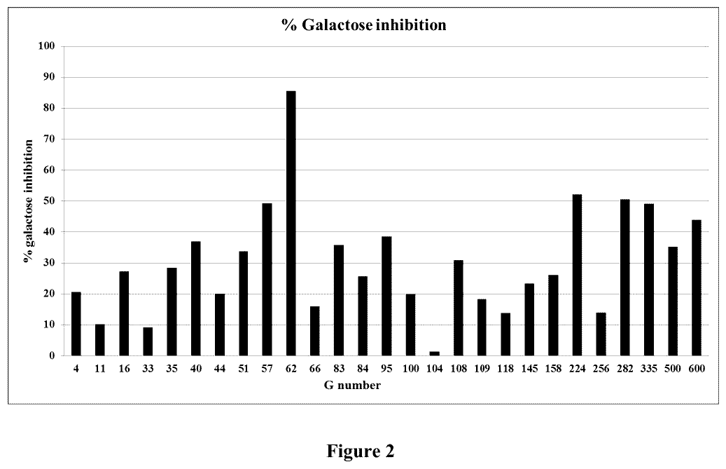

% inhibition=100*(A−B)/A

Where A is specific activity in of enzymes with lactose at pH 6.7 at 37° C. (SUAL) as described in the example 6, and B stand for the specific activity of enzymes in presence of galactose at pH 6.7 at 37° C. (SUAG) as described in the example 7. The detail results of the % galactose inhibition are described FIG. 2 and FIG. 28. The lower galactose inhibition is highly relevant for the applications where very low lactose concentration is desired.

Additionally the activity was described as μmole of glucose formed per minute per milligram of enzyme added. The results are shown in FIG. 28.

Note: relatively high standard deviations in galactose inhibition measurement are due to trace amounts of glucose impurities in purchased galactose.

### Example 8

### Activity Determination Using Purified Enzymes on Lactose as Substrate at pH 6.7 at 4° C.

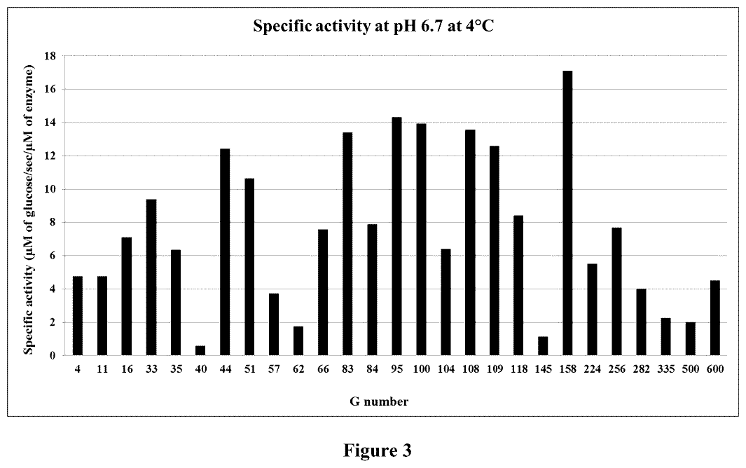

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer B (140 mM of lactose prepared in 100 mM sodium-citrate buffer of pH 6.7, containing 100 μM of MgSO₄). The reaction mixture was prepared by mixing 13 μL of diluted purified enzyme and 37 μL of lactose solution in a PCR tube. The reaction mixture was incubated in a DNA thermal cycler using the following incubating parameters (reaction time; 60 min at 4° C., enzyme inactivation; 10 min at 95° C., storage; 4° C.). The reaction mixtures were stored at-20° C. freezer until further use. The amount of glucose formed during the reaction was determined by following the protocol described in example 6. The maximum absorbance value for each lactase was used to determine UM of glucose formed per sec, described as 1 Unit of Activity with Lactose at pH 6.7 at 4° C. (UAL-2). The specific activity at pH 6.7 at 4° C. is defined as UM of glucose formed per second per μM of enzyme (μM of glucose/sec/μM of enzyme), and is determined by dividing UAL-2 by the protein concentration in μM, described as SUAL-2. The high specific activity at pH 6.7 at 4° C. is highly desired for the lactose hydrolysis for fresh/pasteurized milk applications. The detail results of the specific activity of enzymes at pH 6.7 at 4° C. are described in FIG. 3.

Additionally the activity was described as μmole of glucose formed per minute per milligram of enzyme added. The results are shown in FIG. 28.

### Example 9

### Activity Determination Using Purified Enzymes on Lactose as Substrate at pH 6.7 at 43° C.

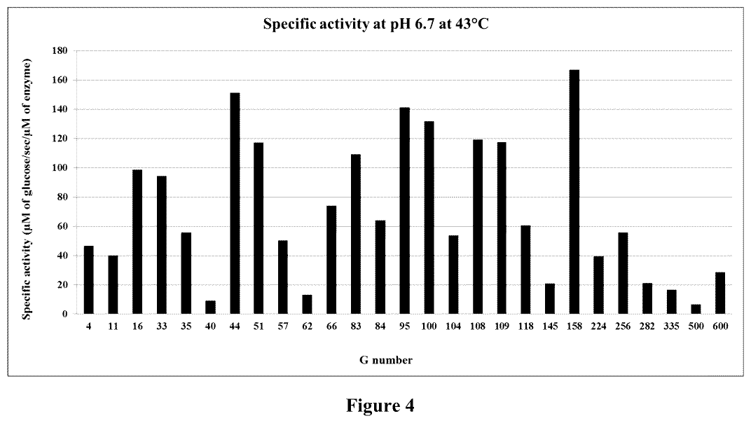

The purified lactases were diluted to 40× in buffer A (50 mM NaH₂PO₄ buffer pH 6.7 containing 100 μM of MgSO₄). In a separate reaction, the diluted enzyme was incubated with lactose solution prepared in buffer B (140 mM of lactose prepared in 100 mM sodium-citrate buffer of pH 6.7, containing 100 μM of MgSO₄). The reaction mixture was prepared by mixing 13 μL of diluted purified enzyme and 37 μL of lactose solution in a PCR tube. The reaction mixture was incubated in a DNA thermal cycler using the following incubating parameters (reaction time; 10 min at 43° C., enzyme inactivation; 10 min at 95° C., storage; 4° C.). The reaction mixtures were stored at −20° C. freezer until further use. The amount of the glucose formed during the reaction was determined by following the protocol described in example 6. The maximum absorbance value for each lactase was used to determine μM of glucose formed per sec, described as 1 Unit of Activity with Lactose at pH 6.7 at 43° C. (UAL-3). The specific activity at pH 6.7 at 43° C. is defined as μM of glucose formed per second per μM of enzyme (μM of glucose/sec/μM of enzyme), and is determined by dividing UAL-3 by the protein concentration in μM, described as SUAL-3. The high specific activity at pH 6.7 at 43° C. is highly desired for the lactose hydrolysis for the fermented milk applications. The detail results of the specific activity of enzymes at pH 6.7 at 43° C. are described in FIG. 4.

Additionally the activity was described as μmole of glucose formed per minute per milligram of enzyme added. The results are shown in FIG. 28.

### Example 10

### Activity Determination Using Purified Enzymes on Lactose as Substrate at pH 5.5 at 4° C.

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer E (140 mM of lactose prepared in 100 mM sodium-citrate buffer of pH 5.5, containing 100 μM of MgSO₄). The reaction mixture was prepared by mixing 13 μL of diluted purified enzyme and 37 μL of lactose solution in a PCR tube. The substrate solution was prepared in a buffer of pH 5.5 and enzyme solution had a pH of 6.7. To initiate the reaction, 13 μL of enzyme was added to 37 μL of substrate solution. This mixing of these two buffers eventually increases the reaction pH from 5.5 to 5.7.

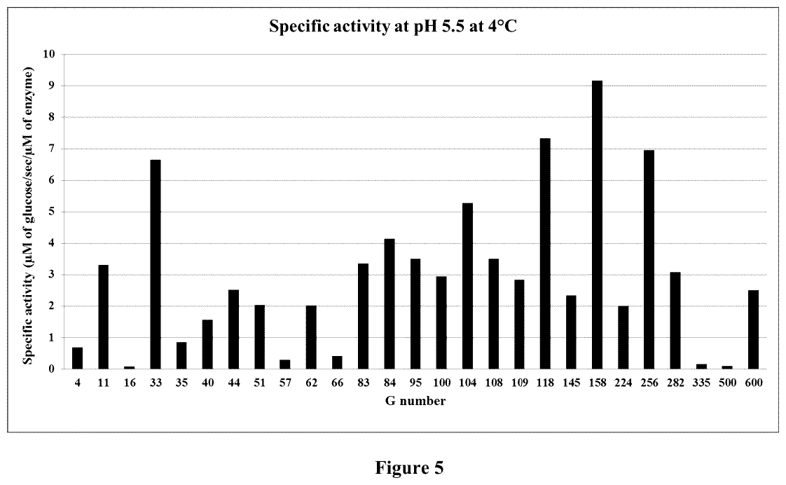

The reaction mixture was incubated in a DNA thermal cycler using the following incubating parameters (reaction time; 60 min at 4° C., enzyme inactivation; 10 min at 95° C., storage; 4° C.). The reaction mixtures were stored at −20° C. freezer until further use. To determine the amount of glucose formed during the reaction, 10 μL of the reaction mixture was transferred to one well of standard microtiter plate containing 80 μL of buffer C and incubated at 30° C. for 40 min. After 40 min, the absorbance was determined at 610 nm using FLUOStar Omega UV-plate reader (BMG Labtech, Germany). The absorbance value between 0.1 and 1.5 were used for calculations, if the A610 nm value>1.5, the reaction mixture was diluted up to 5× with buffer A. With each purified enzyme, the reactions were carried out in triplicate and the mean value of the triplicate measurement was used for calculations. The maximum absorbance value for each lactase was used to determine μM of glucose formed per sec, described as 1 Unit of Activity with Lactose at pH 5.5 at 4° C. (UAL-4). The specific activity at pH 5.5 at 4° C. is defined as UM of glucose formed per second per UM of enzyme (μM glucose/sec/μM of enzyme), and is determined by dividing UAL-4by the protein concentration in μM, described as SUAL-4. The high specific activity at pH 5.5 at 4° C. is relevant for the lactose hydrolysis in the fermented milk applications. The detail results of the specific activity of enzymes at pH 5.5 at 4° C. are described in FIG. 5.

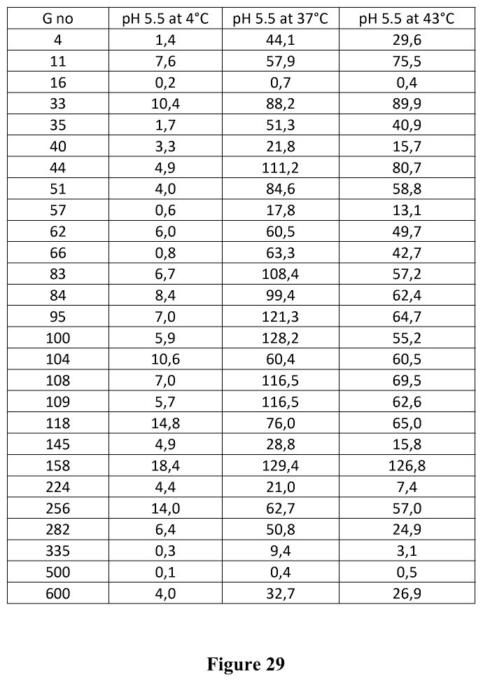

Additionally the activity was described as μmole of glucose formed per minute per milligram of enzyme added. The results are shown in FIG. 29.

### Example 11

### Activity Determination Using Purified Enzymes on Lactose as Substrate at pH 5.5 at 37° C.

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer E (140 mM of lactose prepared in 100 mM sodium-citrate buffer of pH 5.5, containing 100 μM of MgSO₄). The reaction mixture was prepared by mixing 13 μL of diluted purified enzyme and 37 μL of lactose solution in a PCR tube. The substrate solution was prepared in a buffer of pH 5.5 and enzyme solution had a pH of 6.7. To initiate the reaction, 13 μL of enzyme was added to 37 μL of substrate solution. This mixing of these two buffers eventually increases the reaction pH from 5.5 to 5.7.

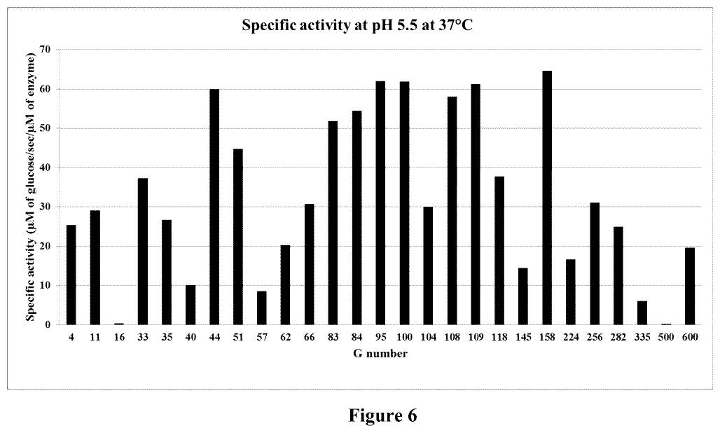

The reaction mixture was incubated in a DNA thermal cycler using the following incubating parameters (reaction time; 10 min at 37° C., enzyme inactivation; 10 min at 95° C., storage; 4° C.). The reaction mixtures were stored at-20° C. until further use. The amount of glucose formed during the reaction was determined by following the protocol as described in the example 10. The maximum absorbance value for each lactase was used to determine μM of glucose formed per sec, described as 1 Unit of Activity with Lactose at pH 5.5 at 37° C. (UAL-5). The specific activity at pH 5.5 at 37° C. is defined as μM of glucose formed per second per μM of enzyme (μM of glucose/sec/μM of enzyme), and is determined by dividing UAL-5 by the protein concentration in μM, described as SUAL-5. The high specific activity at pH 5.5 at 37° C. is relevant for the lactose hydrolysis in the fermented milk applications and sweet whey lactose hydrolysis. The detail results of the specific activity of enzymes at pH 5.5 at 37° C. are described in FIG. 6.

Additionally the activity was described as μmole of glucose formed per minute per milligram of enzyme added. The results are shown in FIG. 29.

### Example 12

### Activity Determination Using Purified Enzymes on Lactose as Substrate at pH 5.5 at 43° C.

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer E (140 mM of lactose prepared in 100 mM sodium-citrate buffer of pH 5.5, containing 100 μM of MgSO₄). The reaction mixture was prepared by mixing 13 μL of diluted purified enzyme and 37 μL of lactose solution in a PCR tube. The substrate solution was prepared in a buffer of pH 5.5 and enzyme solution had a pH of 6.7. To initiate the reaction, 13 μL of enzyme was added to 37 μL of substrate solution. This mixing of these two buffers eventually increases the reaction pH from 5.5 to 5.7.

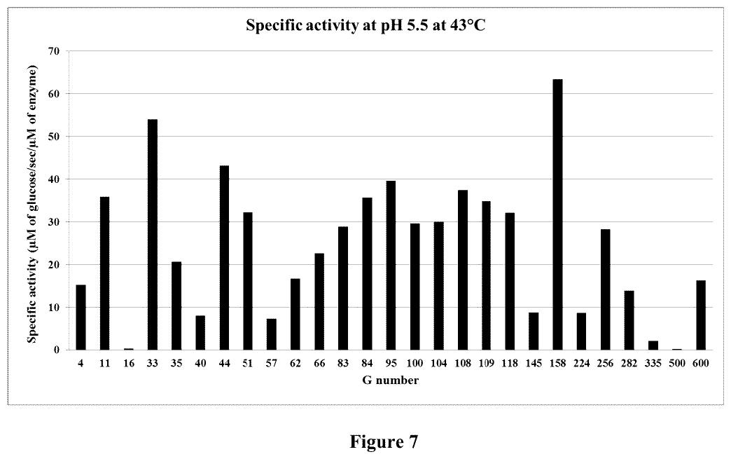

The reaction mixture was incubated in a DNA thermal cycler using the following incubating parameters (reaction time; 10 min at 43° C., enzyme inactivation; 10 min at 95° C., storage; 4° C.). The reaction mixtures were stored at −20° C. until further use. The amount of glucose formed during the reaction was determined by following the protocol described in the example 10. The maximum absorbance value for each lactase was used to determine μM of glucose formed per sec, described as 1 Unit of Activity with Lactose at pH 5.5 at 43° C. (UAL-6). The specific activity at pH 5.5 at 43° C. is defined as μM of glucose formed per second per μM of enzyme (μM of glucose/sec/μM of enzyme), and is determined by dividing UAL-6 by the protein concentration in μM, described as SUAL-6. The high specific activity at pH 5.5 at 43° C. is relevant for the lactose hydrolysis in the fermented milk applications and sweet whey lactose hydrolysis. The detail results of the specific activity of enzymes at pH 5.5 at 43° C. are described in FIG. 7.

Additionally the activity was described as μmole of glucose formed per minute per milligram of enzyme added. The results are shown in FIG. 29.

### Example 13

### Activity Determination Using Purified Enzymes on Lactose as Substrate at pH 4.5 at 4° C.

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer F (140 mM of lactose prepared in 100 mM sodium-citrate buffer of pH 4.5, containing 100 μM of MgSO₄). The reaction mixture was prepared by mixing 13 μL of diluted purified enzyme and 37 μL of lactose solution in a PCR tube. The substrate solution was prepared in a buffer of pH 4.5 and enzyme solution had a pH of 6.7. To initiate the reaction, 13 μL of enzyme was added to 37 μL of substrate solution. This mixing of these two buffers eventually increases the reaction pH from 4.5 to 4.7.

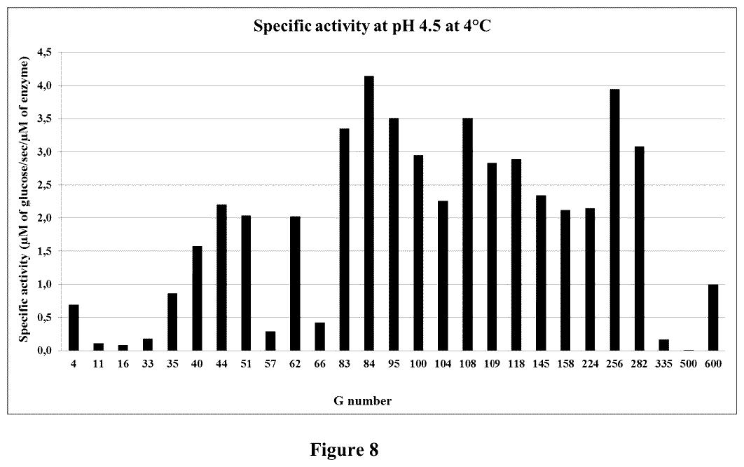

The reaction mixture was incubated in a DNA thermal cycler using the following incubating parameters (reaction time; 60 min at 4° C., enzyme inactivation; 10 min at 95° C., storage; 4° C.). To determine the amount of glucose formed during the reaction, 10 μL of the reaction mixture was transferred to one well of standard microtiter plate containing 80 μL of buffer C (as described in example 6) and incubated at 30° C. for 40 min. After 40 min, the absorbance was determined at 610 nm using FLUOStar Omega UV-plate reader. The absorbance value between 0.1 and 1.5 were used for calculations, if the A610 nm value>1.5, the reaction mixture was diluted up to 5× with buffer A. With each purified enzyme, the reactions were carried out in triplicate and the mean value of the triplicate measurement was used for calculation. The maximum absorbance value for each lactase was used to determine UM of glucose formed per sec, described as 1 Unit of Activity with Lactose at pH 4.5 at 4° C. (UAL-7). The specific activity at pH 4.5 at 4° C. is defined as μM of glucose formed per second per UM of enzyme (UM of glucose/sec/μM of enzyme), and is determined by dividing UAL-7 by the protein concentration in μM, described as SUAL-7. The high specific activity at pH 4.5 at 4° C. is relevant for the lactose hydrolysis in the fermented milk applications. The detail results of the specific activity of enzymes at pH 4.5 at 4° C. are described in the FIG. 8.

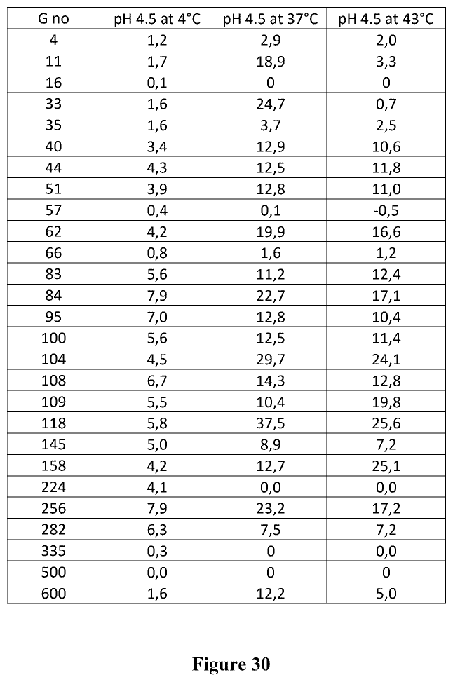

Additionally the activity was described as μmole of glucose formed per minute per milligram of enzyme added. The results are shown in FIG. 30.

### Example 14

### Activity Determination Using Purified Enzymes on Lactose as Substrate at pH 4.5 at 37° C.

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer F (140 mM of lactose prepared in 100 mM sodium-citrate buffer of pH 4.5, containing 100 μM of MgSO₄). The reaction mixture was prepared by mixing 13 μL of diluted purified enzyme and 37 μL of lactose solution in a PCR tube. The substrate solution was prepared in a buffer of pH 4.5 and enzyme solution had a pH of 6.7. To initiate the reaction, 13 μL of enzyme was added to 37 μL of substrate solution. This mixing of these two buffers eventually increases the reaction pH from 4.5 to 4.7.

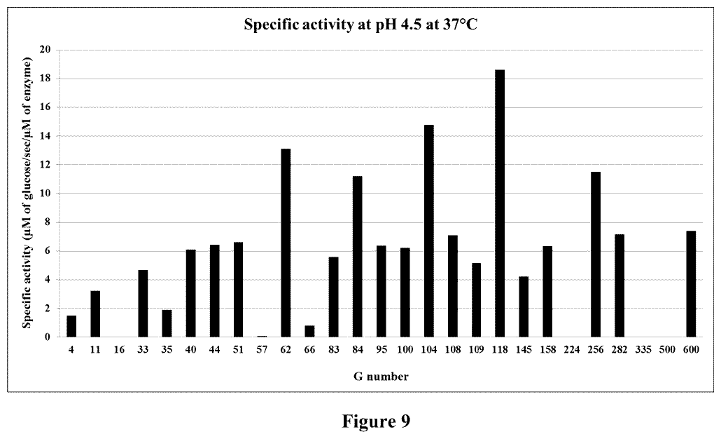

The reaction mixture was incubated in a DNA thermal cycler using the following incubating parameters (reaction time; 10 min at 37° C., enzyme inactivation; 10 min at 95° C., storage; 4° C.). The reaction mixtures were stored at-20° C. until further use. The amount of glucose formed during the reaction was determined by following the protocol described in the example 13. The maximum absorbance value for each lactase was used to determine UM of glucose formed per sec, described as 1 Unit of Activity with Lactose at pH 4.5 at 37° C. (UAL-8). The specific activity at pH 4.5 at 37° C. is defined as UM of glucose formed per second per UM of enzyme (μM of glucose/sec/μM of enzyme), and is determined by dividing UAL-8 by the protein concentration in μM, described as SUAL-8. The high specific activity at pH 4.5 at 37° C. is relevant for the lactose hydrolysis in the fermented milk applications and acidic whey lactose hydrolysis. The detail results of the specific activity of enzymes at pH 4.5 at 37° C. are described in FIG. 9. Additionally the activity was described as μmole of glucose formed per minute per milligram of enzyme added. The results are shown in FIG. 30.

### Example 15

### Activity Determination Using Purified Enzymes on Lactose as Substrate at pH 4.5 at 43° C.

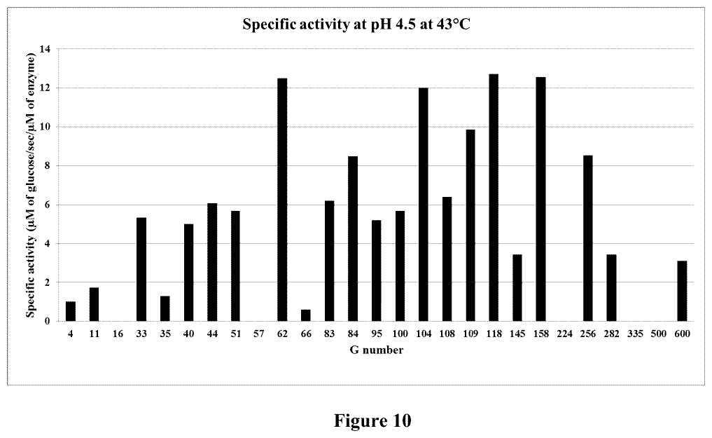

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer F (140 mM of lactose prepared in 100 mM sodium-citrate buffer of pH 4.5, containing 100 μM of MgSO₄). The reaction mixture was prepared by mixing 13 μL of diluted purified enzyme and 37 μL of lactose solution in a PCR tube. The substrate solution was prepared in a buffer of pH 4.5 and enzyme solution had a pH of 6.7. To initiate the reaction, 13 μL of enzyme was added to 37 μL of substrate solution. This mixing of these two buffers eventually increases the reaction pH from 4.5 to 4.7. The reaction mixture was incubated in a DNA thermal cycler using the following incubating parameters (reaction time; 10 min at 43° C., enzyme inactivation; 10 min at 95° C., storage; 4° C.). The reaction mixtures were stored at −20° C. until further use. The amount of glucose formed during the reaction was determined by following the protocol described in the example 13. The maximum absorbance value for each lactase was used to determine μM of glucose formed per sec, described as 1 Unit of Activity with Lactose at pH 4.5 at 43° C. (UAL-9). The specific activity at pH 4.5 at 43° C. is defined as UM of glucose formed per second per μM of enzyme (μM of glucose/sec/μM of enzyme), and is determined by dividing UAL-9 by the protein concentration in μM, described as SUAL-9. The high specific activity at pH 4.5 at 43° C. is relevant for the lactose hydrolysis in the fermented milk applications and acidic whey lactose hydrolysis. The detail results of the specific activity of enzymes at pH 4.5 at 43° C. are described in FIG. 10.

Additionally the activity was described as μmole of glucose formed per minute per milligram of enzyme added. The results are shown in FIG. 30.

### Example 16

### Activity Determination in BLU Units

The commercially available NOLA® Fit enzyme (Chr-Hansen, Denmark) was diluted in a range from 0.5 BLU/mL to 2.5 BLU/mL in buffer G (50 mM NaH₂PO₄ buffer pH 7.0 containing 100 μM of MgSO₄, 0.045% Brij, Sigma Aldrich). The diluted enzyme was incubated with lactose solution prepared in buffer H (105 mM of lactose prepared in 100 mM sodium-citrate buffer of pH 6., containing 100 μM of MgSO₄). The reaction mixture was prepared by mixing 13 μL of diluted purified enzyme and 37 μL of lactose solution in a PCR tube. The reaction mixture was incubated in a DNA thermal cycler using the following incubating parameters (reaction time; 10 min at 37° C., enzyme inactivation; 10 min at 95° C., storage; 4° C.). The amount of glucose conversion was determined by transferring 10 μL of the reaction mixture in a single well of standard microtiter plate containing 80 μL of buffer C and incubated at 30° C. for 40 min. After 40 min, the absorbance was determined at 610 nm using FLUOStar Omega UV-plate reader (BMG Labtech, Germany). The measured absorbance values were used to draw a standard curve against BLU/mL. The maximum slope of the curve was used to determine the activity of new enzymes in BLU/mL.

### Example 17

### Activity Determination of New Lactases in BLU/mL Using Lactose as Substrate

The purified lactases were diluted up to 50× in buffer A (50 mM NaH₂PO₄ buffer pH 6.7 containing 100 μM of MgSO₄). In a separate reaction, the diluted enzyme was incubated with lactose solution prepared in buffer H (105 mM of lactose prepared in 100 mM sodium-citrate buffer of pH 6.7, containing 100 μM of MgSO₄). The reaction mixture was prepared by mixing 13 μL of diluted purified enzyme and 37 μL of lactose solution in a PCR tube. The reaction mixture was incubated in a DNA thermal cycler using the following incubating parameters (reaction time; 10 min at 37° C., enzyme inactivation; 10 min at 95° C., storage; 4° C.). After the reaction, 10 μL of the reaction mixture was transferred to one well of standard microtiter plate containing 80 μL of buffer C (as described in example 6) and incubated at 30° C. for 40 min. After 40 min, the absorbance was determined at 610 nm using FLUOStar Omega UV-plate reader. The absorbance value between 0.1 and 1.5 were used for calculations, if the A610 nm value>1.5, the reaction mixture was diluted up to 5× with buffer A. The maximum absorbance values were used to calculate the enzyme activity in BLU/mL, using standard curve described in example 16.

### Example 18

### Percentage Residual Lactose Measurement in Fresh Milk at Cold Temperature

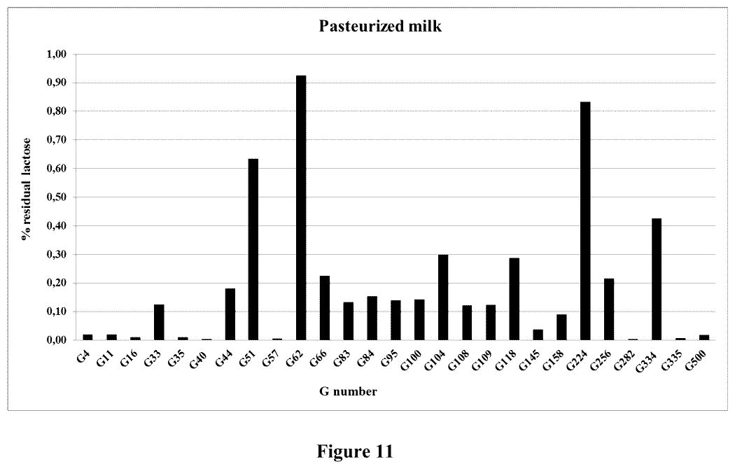

2 mL of commercial pasteurized milk (1.5% Fat pasteurized milk, Arla Food) was mixed with 10-125 μL of enzyme (equivalent to 10 BLU/mL) as determined in the example 17, in 10 mL glass tube. The samples were incubated under constant conditions for 24 hours at 4° C. After the incubation, the reaction was stopped by heat inactivation at 95° C. for 7 min, followed by storage at −20° C. until further use. The amount of remaining lactose in the milk was analyzed using an HPLC assay. Samples for analysis were treated with 1.8 mL protein precipitation solution (0.083 M PCA and 2 mM Na-EDTA) and 2 mL of MQW prior to centrifugation at 2800 rpm for 30 min at 4° C. An aliquot of the supernatant was diluted a total of 200-fold using a Janus dilution robot (PerkinElmer, Waltham, MA, USA). The diluted samples were analyzed on a Dionex ICS-5000 system (Thermo Fischer Scientific, Waltham (MA), USA) using 4×250 mm CarboPac SA20 analytical column (Thermo Fischer Scientific, Waltham, MA, USA) and a pulsed amperometric detector. The detector was set to a simple three-step potential waveform, selective for detection of carbohydrates. The eluent was set to 1 mM KOH and was continuously regenerated through a trap column (CR-TC, Thermo Fischer Scientific, Waltham (MA), USA). The flow rate of the eluent was 1.2 mL/min and the analysis time was 10 min per injection. The lactose in each sample was quantified using a three-point external calibration curve prepared by adding known amounts of lactose monohydrate (Sigma-Aldrich, St. Louis, MO, USA) to MQW. Concentrations were calculated based on the chromatographic peak heights. The measured percentage residual lactose in fresh milk is shown in FIG. 11.

### Example 19

### Activity Determination in UHT Milk at Room Temperature

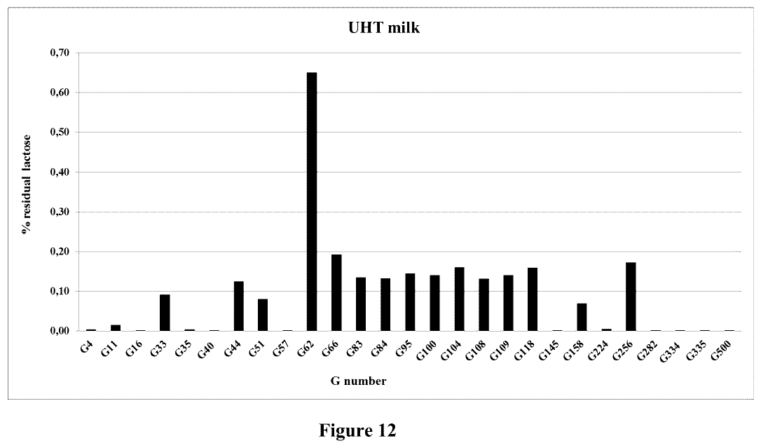

2 mL of UHT milk (1.5% Fat UHT milk, Arla Food) was mixed with 2-25 μl of enzyme (equivalent to 2 BLU/mL) as determined in example 17, in 10 mL glass tube. The samples were incubated under constant conditions for 24 hours at 25° C. After the incubation, the reaction was stopped by heat inactivation at 95° C. for 7 min, followed by storage at −20° C. until further use. The amount of residual lactose in UHT milk was analyzed using HPLC by following the protocol as described in example 18. The percentage of residual lactose in fresh milk after hydrolysis is listed in FIG. 12.

### Example 20

### Enzyme Performance at High Temperature in Buffer

The purified enzyme was diluted to 5 BLU/mL in buffer A (50 mM NaH₂PO₄ buffer pH 6.7 containing 100 μM of MgSO₄). In a separate reaction, 13 μL of the diluted enzyme was incubated in a DNA thermal cycler with lactose solution (105 mM lactose prepared in 100 mM sodium-citrate buffer of pH 6.7, containing 100 μM of MgSO₄). The reaction mixture was prepared by mixing 13 μl of enzyme and 37 μL of lactose solution in a PCR tube. The reaction mixture was incubated in a DNA thermal cycler using the following incubating parameters (reaction time; 10 min at 37° C., enzyme inactivation; 10 min at 95° C., storage; 4° C.). After the reaction, 10 μL of the reaction mixture was transferred to one well of standard microtiter plate containing 80 μL of buffer C (as described in example 6) and incubated at 30° C. for 40 min. After 40 min, the absorbance was determined at 610 nm using FLUOStar Omega UV-plate reader. The absorbance value between 0.1 and 1.5 were used for calculations, if the A610 nm value>1.5, the reaction mixture was diluted up to 5× with buffer A. The measured absorbance was called Abs37° C., and considered as reference value for calculations.

To measure the impact of heat treatment on enzyme activity, in a separate reaction, 13 μL of the diluted enzyme (5 BLU/mL) was incubated in a DNA thermal cycler using the following incubating parameter (at 72° C. for 15 sec or 74° C. for 15 sec or 76° C. for 6 sec or 78° C. for 6 sec or 80° C. for 4 sec or 85° C. for 5 sec or 90° C. for 5 sec or 95° C. for 5 sec, followed by storage at 4° C.). The activity of the heat treated enzyme was determined by incubation with the lactose solution (105 mM lactose prepared in 100 mM sodium-citrate buffer of pH 6.7, containing 100 μM of MgSO₄), as described above. The measured absorbance at different temperature (for example at 72° C., 74° C., 76° C., 78° C., 80° C., 85° C., 90° C. or 95° C.) was called as Abs72° C., Abs74° C., Abs76° C., Abs78° C., Abs80° C., Abs85° C., Abs90° C., Abs95° C.

The percentage residual activity at high temperature was determined using the formula,

% residual activity=(Abs72° C./Abs37° C.)*100

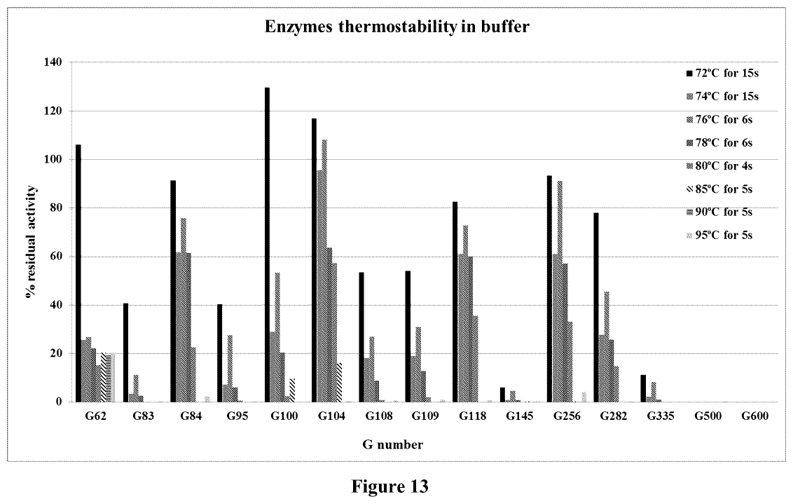

The percentages residual activities of different enzymes at different temperature are described in FIG. 13.

### Example 21

### Percentage Residual Lactose After the High Heat Treatment

The effect of heat treatment on the enzyme performance in pasteurized milk was determined by incubating a fixed amount of enzyme in the milk followed by a heat treatment. In separate reactions, 50 μL of the pasteurized milk was mixed with 10 BLU/mL of purified enzyme (as determined in example 17), in a PCR tube. The milk sample was incubated at 72° C. for 15 or 76° C. for 10 sec or 85° C. for 5 sec and 90° C. for 5 sec, followed by incubation at 5° C. for 24 h. After 24 h at 5° C., the reaction was stopped by heating the reaction at 95° C. for 7 min, followed by storage at −20° C. The residual lactose was measured by using the LactoSens® assay kit (Chr. Hansen, Denmark), by following the supplied protocol. The measured residual lactose was determined in g/L was determined at different temperature. The detection limit of the LactoSens® kit is between 0.2 g/L to 10 g/L. The results are described in the table 3:

Table 3: The percentage residual lactose in the pasteurized milk treated with a fixed amount of the purified enzyme followed by incubation at different temperature (72° C. for 15 sec, 76° C. for 10 sec, 85° C. for 5 sec and 90° C. for 5 sec followed by incubation at 4C for 24 h), determined using LactoSens® assay kit. The LactoSens® kit detection limits are in range of 0.2 g/L to 10 g/L of lactose. Here ND; not determined.

TABLE 3

| | Residual lactose at | | | | |
|---|---|---|---|---|---|
| G-No. | 4° C. (g/L) | 72° C. (g/L) | 76° C. (g/L) | 85° C. (g/L) | 90° C. (g/L) |
| G4 | <0.2 | >10.0 | ND | ND | ND |
| G11 | <0.2 | >10.0 | ND | ND | ND |
| G16 | <0.2 | >10.0 | ND | ND | ND |
| G33 | <0.2 | 4.7 | ND | ND | ND |
| G35 | <0.2 | >10.0 | >10.0 | ND | ND |
| G40 | <0.2 | <0.2 | <0.2 | >10.0 | ND |
| G44 | 0.9 | >10.0 | ND | ND | ND |
| G57 | <0.2 | >10.0 | ND | ND | ND |
| G62 | 8.4 | >10.0 | >10.0 | >10.0 | ND |
| G66 | 0.35 | >10.0 | ND | ND | ND |
| G83 | 0.3 | 2.1 | 6.0 | >10.0 | ND |
| G84 | 0.25 | 0.65 | 0.5 | 7.6 | >10 |
| G95 | 0.3 | 6.0 | 8.6 | >10 | ND |
| G100 | 0.4 | 2.4 | 2.6 | >10.0 | ND |
| G104 | 0.35 | 0.45 | 0.5 | 0.45 | >10 |
| G108 | 0.35 | 1.3 | 1.55 | ND | ND |
| G109 | 0.35 | 1.45 | 3.4 | >10.0 | ND |
| G118 | 0.45 | 0.95 | 0.8 | >10.0 | >10 |
| G158 | <0.2 | 3.9 | >10.0 | ND | ND |
| G256 | 0.3 | 1.0 | 0.75 | 3.4 | >10 |
| G282 | <0.2 | <0.2 | <0.2 | <0.2 | >10 |
| G335 | <0.2 | 0.35 | 8.0 | >10.0 | ND |
| G600 | <0.2 | >10.0 | >10.0 | >10.0 | ND |
| G500 | <0.2 | >10.0 | ND | ND | ND |

### Example 22

### Percentage Residual Lactose in Pasteurized Milk Incubated at Different Temperatures

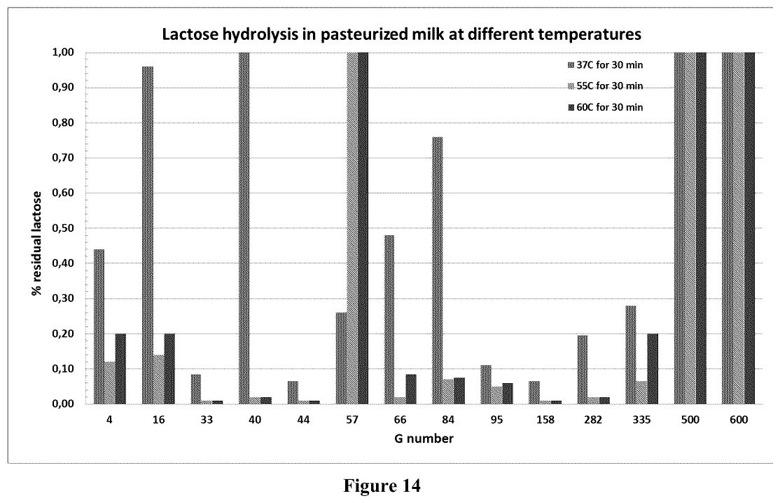

1 mL of commercial pasteurized milk (1.5% fat milk containing 4.7% lactose, Arla Foods, Denmark) was mixed with 0.047 mg/ml of enzyme, in a 1.5 mL Eppendorf tube. The enzyme was mixed in the milk with gentle vortex or pipetting. 50 μL of the milk, containing the enzyme, was transferred to a PCR tube. For each enzyme the reaction was performed in 2×50 μl reaction volume. The reaction mixture was incubated in a DNA thermal cycler with the following incubation parameters (reaction temperatures and time; 37° C. for 30 min or 55° C. for 30 min or 60° C. for 30 min, enzyme inactivation temperature and time; 95° C. for 10 min, storage temperature: 4° C.). During the enzyme addition, pipetting and mixing the milk samples were kept on ice-water mixture to minimize the effect of temperature on enzyme performance. After the reaction, the milk samples were either used directly for the residual lactose measurement or stored at −20° C. until further use. The residual lactose in the milk was analyzed using LactoSens® assay kit (Chr. Hansen, Denmark) by following the protocol supplied with the kit. The measured percentage residual lactose in the pasteurized milk is shown in FIG. 14.

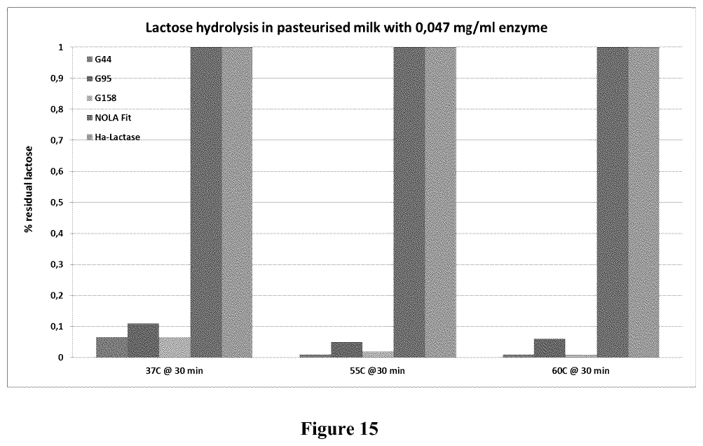

To test the lactose hydrolysis potential of these novel lactases, we incubated 0.047 mg enzyme per milliliter of the pasteurized milk and incubated at 37° C., 55° C. and 60° C. for 30 min. After 30 min incubation, the enzymes were inactivated by heating at 95° C. The residual lactose was determined using LactoSens® assay kit (Chr. Hansen, Denmark). At their optimal temperature (37° C.), both the Ha-Lactase and NOLA® fit showed a high residual lactose (>1% of residual lactose), suggesting that enzymes have lower activity and are not producing lactose free pasteurized milk in the given time frame. Moreover, a similar level of residual lactose was measured at 55° C. and 60° C. On the contrary, the G33, G44, G95 and G158 enzymes showed <0.1% residual lactose at 37° C., FIG. 15. Because of their high activity at elevated temperatures (55° C. or 60° C.), the novel enzymes showed <0.01% residual lactose after 30 min incubation. This shows that by using the current enzyme dose it is possible to produce essentially lactose free pasteurized and filtered milk in less than 30 min. Filtered milk is more like raw milk than like pasteurized milk. The lactose hydrolysis at elevated temperature (55° C. −60° C.) in short time reduces the chance of microbial growth without affecting the milk quality.

### Example 23

### Percentage Residual Lactose in Pasteurized Milk Incubated for Different Time Span

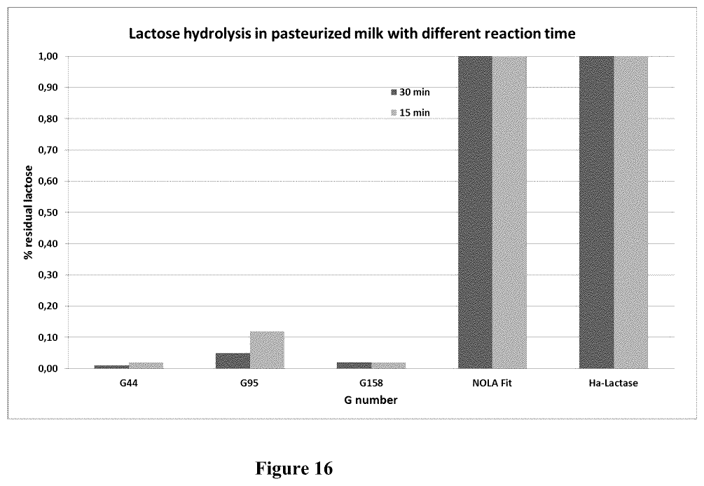

1 mL of commercial pasteurized milk (1.5% fat milk containing 4.7% lactose, Arla Foods, Denmark) was mixed with 0.047 mg/mL of enzyme, in a 1.5 mL Eppendorf tube. The enzyme was mixed in the milk with gentle vortex or pipetting. 50 μL of the milk, containing the enzyme, was transferred to a PCR tube. For each enzyme the reaction was performed in 2×50 μL reaction volume. The reaction mixture was incubated in a DNA thermal cycler with the following incubation parameters (reaction temperatures and time; 55° C. for 15 min or 55° C. for 30 min, enzyme inactivation temperature and time; 95° C. for 10 min, storage temperature: 4° C.). During the enzyme addition, pipetting and mixing the milk samples were kept on ice-water mixture to minimize the effect of temperature and time. After the reaction, the milk samples either used directly for the residual lactose measurement or stored at −20° C. until further use. The residual lactose in the milk was analyzed using LactoSens® assay kit (Chr. Hansen, Denmark), as described in the example 22. The measured percentage residual lactose in the pasteurized milk is shown in FIG. 16.

### Example 24

### Percentage Residual Lactose in Pasteurized Milk Incubated with Different Enzyme Doses

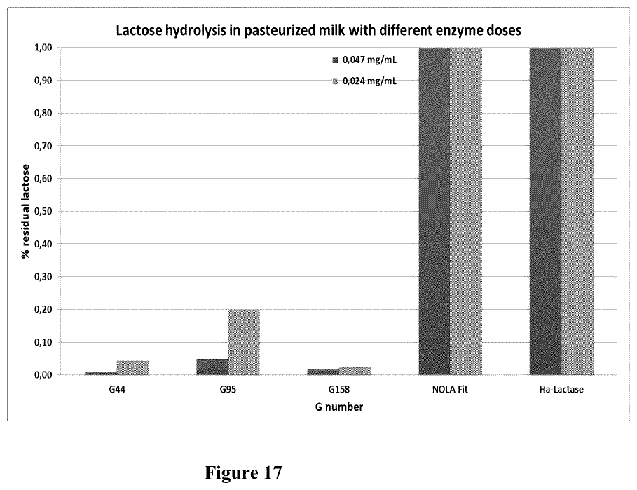

1 mL of commercial pasteurized milk (1.5% fat milk containing 4.7% lactose, Arla Foods, Denmark) was mixed with either different enzyme doses (0.024 mg/ml or 0.047 mg/mL), in 1.5 mL Eppendorf tube. The enzyme was mixed in the milk with gentle vortex or pipetting. 50 μL of the milk, containing the enzyme, was transferred to a PCR tube. For each enzyme the reaction was performed in 2×50 μL reaction volume. The reaction mixture was incubated in a DNA thermal cycler with the following incubation parameters (reaction temperatures and time; 55° C. for 30 min, enzyme inactivation temperature and time; 95° C. for 10 min, storage temperature: 4° C.). After the reaction, the samples either used directly for the residual lactose measurement or stored at −20° C. until further use. The residual lactose in the milk was analyzed by following the same protocol as described in example 22. The measured percentage residual lactose in the pasteurized milk is shown in FIG. 17.

### Example 25

### Percentage Residual Lactose in Pasteurized Milk Incubated with Different Enzyme Doses and for Different Reaction Time Span

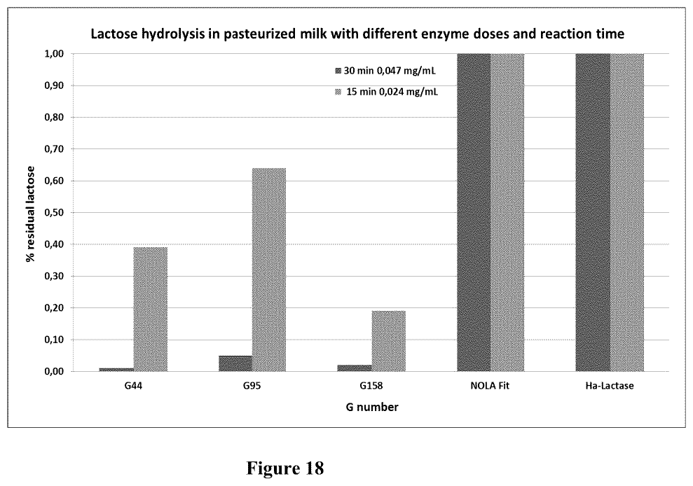

1 mL of commercial pasteurized milk (1.5% fat milk containing 4.7% lactose, Arla Foods, Denmark) was mixed with different enzyme dose (0.024 or 0.047 mg/mL), in 1.5 mL Eppendorf tube. The enzyme was mixed in the milk with gentle vortex or pipetting. 50 μL of the milk, containing the enzyme, was transferred to a PCR tube. For each enzyme the reaction was performed in 2×50 μl reaction volume. The reaction mixture was incubated in a DNA thermal cycler with the following incubation parameters (reaction temperatures and time; 55° C. for 15 min or 55° C. for 30 min, enzyme inactivation temperature and time; 95° C. for 10 min, storage temperature: 4° C.). During the enzyme addition, pipetting and mixing the milk samples were kept on ice-water mixture to minimize the effect of temperature and time. After the reaction, the samples either used directly used the residual lactose measurement or stored at −20° C. until further use. The residual lactose was determined using the protocol described in example 22. The measured percentage residual lactose in the pasteurized milk is shown in FIG. 18.

### Example 26

### Percentage Residual Lactose in Filtered Milk

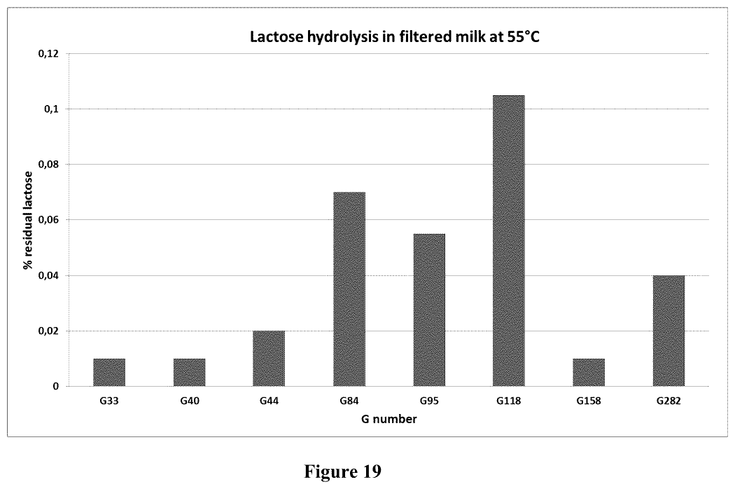

1 mL of commercial micro-filtered semi skimmed milk (1.5% fat milk containing 4.8% lactose, Marguerite, France) was mixed with 0.047 mg/ml of enzyme, in 1.5 mL Eppendorf tube. The enzyme was mixed in the milk with gentle vortex or pipetting. 50 μL of the milk, containing the enzyme, was transferred to a PCR tube. For each enzyme the reaction was performed in 2×50 μL reaction volume. The reaction mixture was incubated in a DNA thermal cycler with the following incubation parameters (reaction temperatures and time; 55° C. for 30 min, enzyme inactivation temperature and time; 95° C. for 10 min, storage temperature: 4° C.). During the enzyme addition, pipetting and mixing the milk samples were kept on ice-water mixture to minimize the effect of temperature and time. After the reaction, the samples either used directly for the residual lactose measurement or stored at −20° C. until further use. The amount of remaining lactose in the milk was analyzed using LactoSens® assay kit (Chr. Hansen, Denmark) by following the protocol supplied with the kit. The measured percentage residual lactose in the filtered milk is shown in FIG. 19.

This shows that by using the current enzyme dose it is possible to produce lactose free filtered milk (filtered milk is more like raw milk than pasteurized) in less than 30 min. The lactose hydrolysis at elevated temperature (55° C.-60° C.) in short time reduces the chance of microbial growth without affecting the milk quality.

### Example 27

### Percentage Residual Lactose in Filtered Milk Incubated with Different Enzyme Doses

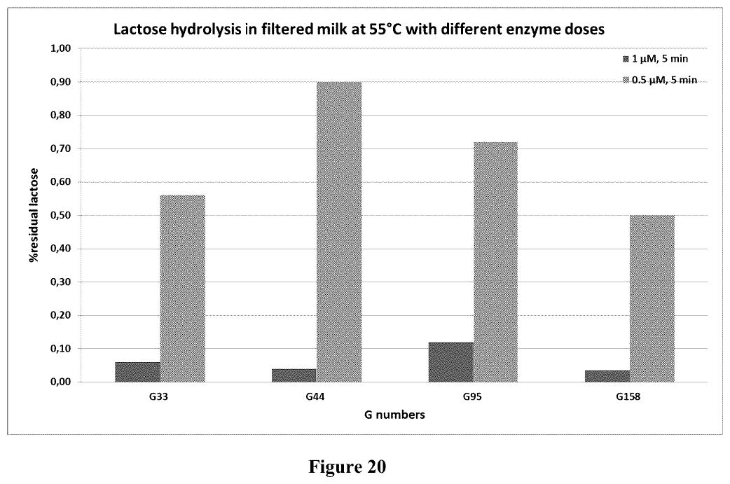

1 ml of commercial micro-filtered semi skimmed milk (1.5% fat milk containing 4.8% lactose, Marguerite, France) was mixed with different enzyme doses (0.055 mg/ml, 0.55 μM or 0.11 mg/ml, 0.11 μM), in 1.5 mL Eppendorf tube. The enzyme was mixed in the milk with gentle vortex or pipetting. 50 μL of the milk, containing the enzyme, was transferred to a PCR tube. For each enzyme the reaction was performed in 2×50 μL reaction volume. The reaction mixture was incubated in a DNA thermal cycler with the following incubation parameters (reaction temperatures and time; 55° C. for 5 min, enzyme inactivation temperature and time; 95° C. for 10 min, storage temperature: 4° C.). During the enzyme addition, pipetting and mixing the milk samples were kept on ice-water mixture to minimize the effect of temperature and time. After the reaction, the samples were either used directly for the residual lactose measurement or stored at −20° C. until further use. The residual lactose in the milk was analyzed by following the protocol described in example 22. The measured percentage residual lactose in the filtered milk is shown in FIG. 20.

### Example 28

### Percentage Residual Lactose in Filtered Milk Incubated for Different Time Span

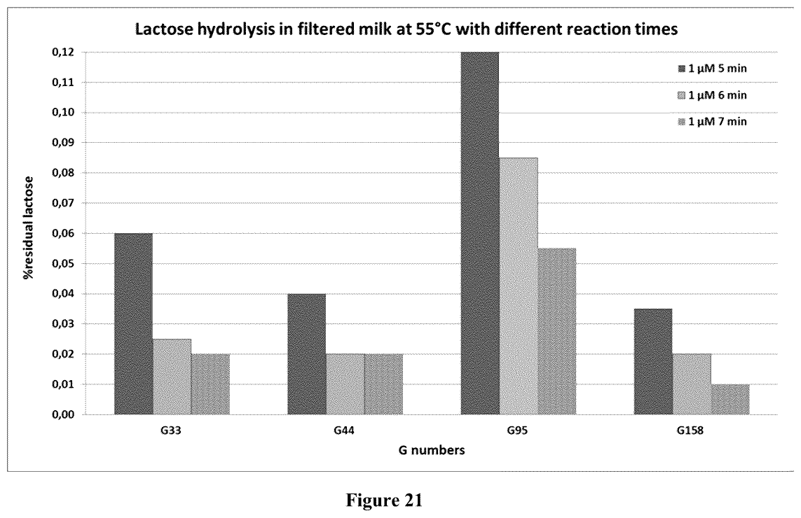

1 mL of commercial micro-filtered semi skimmed milk (1.5% fat milk containing 4.8% lactose, Marguerite, France) was mixed with 0.11 mg/ml (1 μM) of enzyme, in 1.5 mL Eppendorf tube. The enzyme was mixed in the milk with gentle vortex or pipetting. 50 μL of the milk, containing the enzyme, was transferred to a PCR tube. For each enzyme the reaction was performed in 2'50 μL reaction volume. The reaction mixture was incubated in a DNA thermal cycler with the following incubation parameters (reaction temperatures and time; 55° C. for 5 min or 55° C. for 6 min or 55° C. for 7 min, enzyme inactivation temperature and time; 95° C. for 10 min, storage temperature: 4° C.). During the enzyme addition, pipetting and mixing the milk samples were kept on ice-water mixture to minimize the effect of temperature and time. After the reaction, the samples either used directly for residual lactose measurement or stored at −20° C. until further use. The amount of remaining lactose in the milk was analyzed using LactoSens® assay kit (Chr. Hansen, Denmark) by following the protocol supplied with the kit. The measured percentage residual lactose in the filtered milk is shown in FIG. 21. This shows that by using the current enzyme dose it is possible to produce lactose free pasteurized and filtered milk (filtered milk is more like raw milk than pasteurized) in less than 5-30 min. The lactose hydrolysis at elevated temperature (55° C.-60° C.) in short time reduces the chance of microbial growth without affecting the milk quality.

### Example 29

### Enzyme Activity at 4-5° C.

To analyze the kinetics of lactose hydrolysis by the novel enzymes in pasteurized milk, 0.05 mg enzyme was added per milliliter of commercial pasteurized milk (1.5% fat milk containing 4.7% lactose, Arla Foods, Denmark). The enzyme was mixed well by gentle vortex and transferred into PCR tube, 10×100 μL of each. The reaction mixtures were incubated at 4° C., and after a fixed interval the samples was withdrawn. The reaction was stopped by heating at 95° C. for 10 min in PCR machine. The samples were cooled to room temperature and the residual lactose was measured using LactoSens® assay kit (Chr. Hansen, Denmark). The measured value of residual lactose was plotted against reaction time.

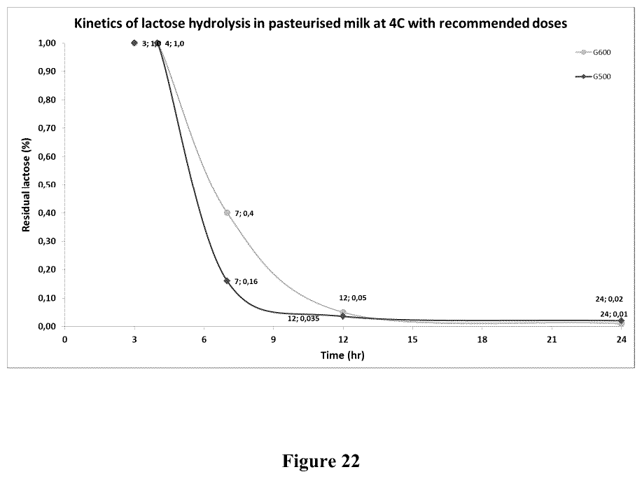

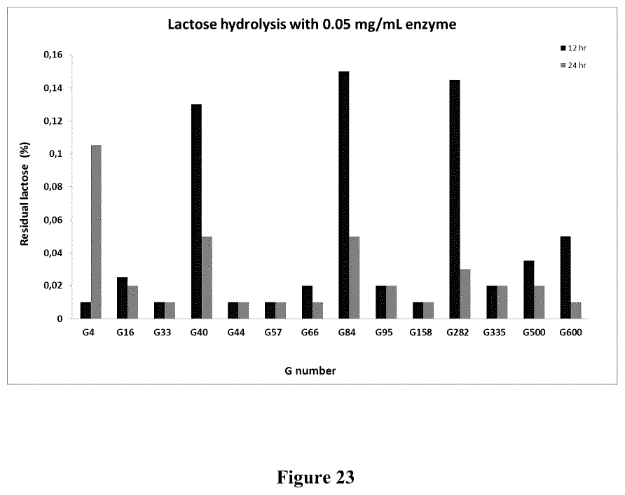

At 4-5° C. the known commercial products, NOLA® Fit (G600) and Ha-Lactase™ (G500) require between 8-12 hr and 18-24 hr to reduce the concentration of residual lactose in cow milk to less than <0.1% and <0.01%, respectively (as shown in FIGS. 22 and 23).

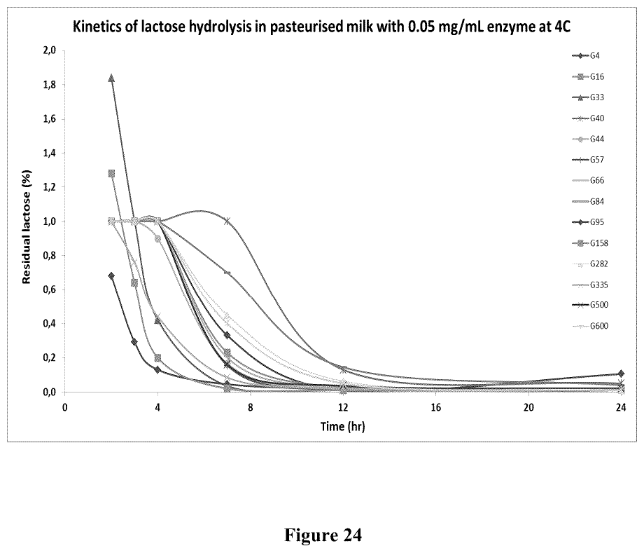

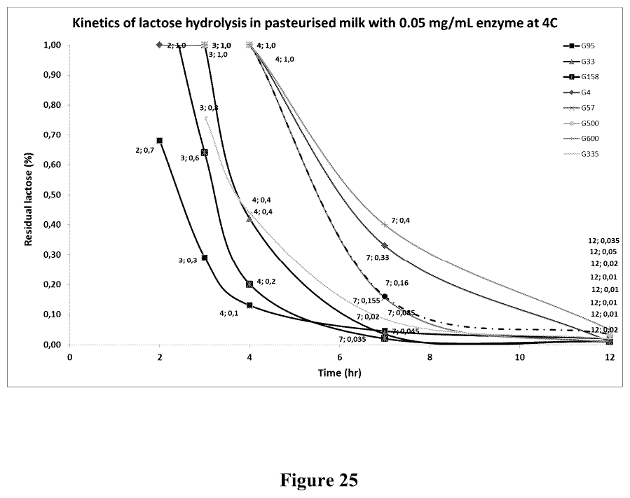

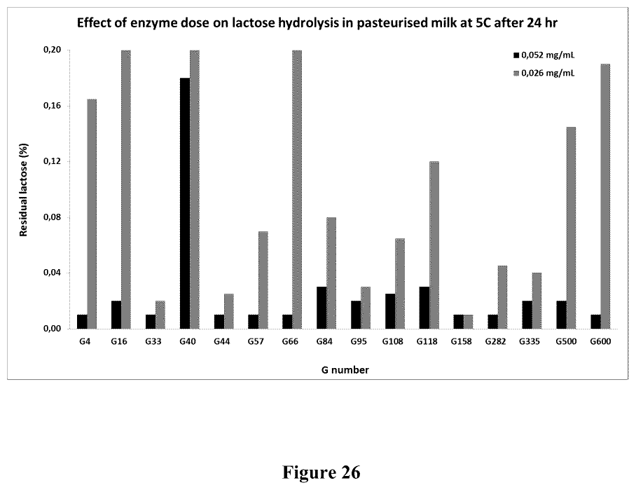

The lactases of the present invention are significantly more active under these conditions. For example, the G95 (the most active enzyme) reaches a residual concentration of lactose of <0.1% level (4 hr). The G158 and G33 are able to reduce the residual concentration of lactose to a level of <0.1% in between 5-6 hr and a level of <0.01% lactose in 8-12 hr. After 12 hr incubation, several of the lactases showed lower residual lactose than control enzymes (shown in FIGS. 24 and 25). These results show that the novel lactases are faster than Ha-Lactase™ and NOLA® Fit and result in lactose free pasteurized milk in significantly shorter time. These new enzymes can reduce the overall process time by 50%. Additionally, the novel enzymes provide the possibility to reduce the enzyme dose further between 25-50% to produce lactose free/reduced pasteurized milk (shown in FIG. 26).

These results thus show that the novel lactases can produce lactose free pasteurized milk in significantly shorter time (8-12 hr) with 50 mg/L enzyme dose. Moreover, it is possible to lower the enzyme dose by 25-50%, depending on the required lactose level.

### Example 30

### Enzyme Activity in Different Milk Types at 4-5° C.

To compare enzyme activity in different milk types, pasteurized and filtered milk was incubated using lactase enzyme in a concentration of 0.052 mg/L. The samples were mixed and stored at 4° C. for 24 hr.

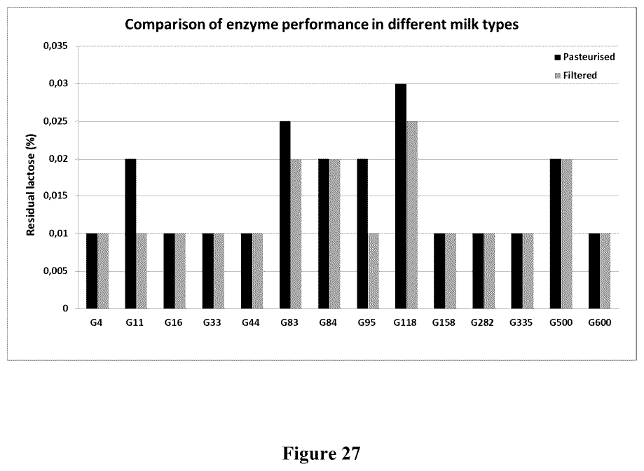

The residual lactose content was determined using LactoSens® assay kit (Chr. Hansen, Denmark) and is shown in FIG. 27, which shows that many of the new lactase enzymes are highly active in digesting lactose in pasteurized and filtered milk at 4° C. Claims

1. A method for producing a dairy product comprising:
(a) mixing a milk-based substrate comprising lactose in a concentration of at least 10 g/L and a peptide or a dimeric peptide exhibiting beta-galactosidase activity in a concentration of 10 to 55 mg/L;
(b) incubating the mixture at a temperature from 1° C.-10° C. for a period of time sufficient to reduce the lactose concentration in the mixture to less than 0.2 g/L.

2. A method for reducing the lactose content in a milk-based substrate comprising:
(a) mixing a milk-based substrate comprising lactose in a concentration of at least 10 g/L and a peptide or a dimeric peptide exhibiting beta-galactosidase activity in a concentration of 10 to 55 mg/L;
(b) incubating the mixture at a temperature from 1° C.-10° C. for a period of time sufficient to reduce the lactose concentration in the mixture to less than 0.2 g/L.

3. Method according to claim 1 or 2, wherein the peptide or dimeric peptide exhibiting beta-galactosidase activity is:
(i) a peptide having an amino acid sequence selected from SEQ ID NO: 22, 33, 14, 7, 9, 11, 30 and 1 or a peptide having an amino acid sequence identity of more than 85% to any of these sequences;
(ii) a peptide having an amino acid sequence selected from SEQ ID NO: 22, 33, 14, 13, 19, 7, 9, 11, 26 and 27, 30 and 1 or a peptide having an amino acid sequence identity of more than 85% to any of these sequences;
(iii) a peptide having an amino acid sequence represented by SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or enzymatically active fragments thereof, or an amino acid sequence of any one thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.

4. Method according to any one of claims 1 to 3, wherein the peptide or dimeric peptide exhibiting beta-galactosidase activity is added in a concentration of 35 to 52 mg/L, in a concentration of 40 to 52 mg/L or in a concentration of 45 to 52 mg/L.

5. Method according to any one of claims 1 to 4, wherein the milk-based substrate comprising lactose is:
(i) cow milk, sheep milk, goat milk, buffalo milk, camel milk, or a pasteurized, raw and/or filtered form thereof; or
(ii) a fermented dairy product obtained from (i) by fermentation.

6. Method according to claim 5, wherein the milk-based substrate comprising lactose is cow milk comprising lactose in a concentration of about 37 to 50 g/L or a heat treated, pasteurized and/or filtered form thereof.

7. Method according to any one of claims 1 to 5, wherein the concentration of less than 0.2 g/l lactose is reached after incubation for at least 4 hours, at least 8 hours, at least 12 hours or at least 24 hours.

8. Method according to any one of claims 1 to 7, wherein the incubation temperature in step (b) is in the range of from 2° C.-7° C. or in the range of from 3° C.-6° C.

9. Method according to any one of claims 1 to 8, wherein the incubation in step (b) reduces the lactose concentration in the mixture to less than 0.05 g/L, to less than 0.02 g/L, or to less than 0.01 g/L.

10. Method according to any one of claims 1 to 9, wherein the mixture comprising the milk-based substrate and the peptide or dimeric peptide exhibiting beta-galactosidase activity is heated to a temperature of at least 60° C. for at least four seconds before or after incubating the mixture at a temperature from 1° C.-10° C.

11. Method according to claim 10, wherein the mixture comprising the milk-based substrate and the peptide or dimeric peptide exhibiting beta-galactosidase activity is heated to a temperature of 72° C. for about 15 seconds before or after incubating the mixture at low temperatures in step (b) or heated to a temperature of 140°° C. for about four seconds before or after incubating the mixture at a temperature from 1° C.-10° C.

12. Method for producing a dairy product according to any one of claims 1 or 3 to 9, wherein the method comprises a step of fermenting the milk-based substrate with lactic acid bacteria.

13. Method according to claim 12, wherein the fermentation step is carried out before or after the incubation with a peptide or dimeric peptide exhibiting beta-galactosidase activity.

14. Method according to any one of claims 1 or 3 to 9, wherein the dairy product is a fermented milk product, cheese, yoghurt, butter, dairy spread, butter milk, acidified milk drink, sour cream, whey based drink, ice cream, condensed milk, dulce de leche or a flavored milk drink.

15. Use of a peptide or dimeric peptide exhibiting beta-galactosidase activity for producing a dairy product with reduced lactose content at a temperature from 1° C.-10° C. for a period of time sufficient to reduce the lactose concentration in the mixture to less than 0.2 g/L, wherein the peptide or dimeric peptide exhibiting beta-galactosidase activity is:
(i) a peptide having an amino acid sequence selected from SEQ ID NO: 22, 33, 14, 7, 9, 11, 30 and 1 or a peptide having an amino acid sequence identity of more than 85% to any of these sequences;
(ii) a peptide having an amino acid sequence selected from SEQ ID NO: 22, 33, 14, 13, 19, 7, 9, 11, 26 and 27, 30 and 1 or a peptide having an amino acid sequence identity of more than 85% to any of these sequences;
(iii) a peptide having an amino acid sequence represented by SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or enzymatically active fragments thereof, or an amino acid sequence of any one thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.
